SEQUENCE LISTING (26 sequences)

SEQ ID NO: 1 - DNA, 30 bp, Artificial Sequence
  Description of Artificial Sequence Synthetic primer
cagccagccagctcctgtacatgagggacc

SEQ ID NO: 2 - DNA, 30 bp, Artificial Sequence
  Description of Artificial Sequence Synthetic primer
gaacctgcgtgcaatccatcttgttcaatg

SEQ ID NO: 3 - DNA, 30 bp, Artificial Sequence
  Description of Artificial Sequence Synthetic primer
tagatggctagaagaatggaggtggattgc

SEQ ID NO: 4 - DNA, 30 bp, Artificial Sequence
  Description of Artificial Sequence Synthetic primer
ggtccctcatgtacaggagctggctggctg

SEQ ID NO: 5 - DNA, 30 bp, Artificial Sequence; identical to SEQ ID NO: 1
  Description of Artificial Sequence Synthetic primer

SEQ ID NO: 6 - DNA, 30 bp, Artificial Sequence
  Description of Artificial Sequence Synthetic primer
cacactggaagtccacctgaaacctggtag

SEQ ID NO: 7 - DNA, 25 bp, Artificial Sequence
  Description of Artificial Sequence Synthetic oligonucleotide
tttacactcaccttcacctcttcgt

SEQ ID NO: 8 - DNA, 25 bp, Artificial Sequence
  Description of Artificial Sequence Synthetic oligonucleotide
acgaagaggtgaaggtgagtgtaaa

SEQ ID NO: 9 - DNA, 25 bp, Artificial Sequence
  Description of Artificial Sequence Synthetic oligonucleotide
tagcggtagtgaagaaagtgctggc

SEQ ID NO: 10 - DNA, 25 bp, Artificial Sequence
  Description of Artificial Sequence Synthetic oligonucleotide
gccagcactttcttcactaccgcta

SEQ ID NO: 11 - DNA, 30 bp, Artificial Sequence
  Description of Artificial Sequence Synthetic primer
ctacccttgcaatacgaggacaaagaagtg

SEQ ID NO: 12 - DNA, 28 bp, Artificial Sequence
  Description of Artificial Sequence Synthetic primer
tcaggatgtcatgttaccatttgtgatg

SEQ ID NO: 13 - DNA, 27 bp, Artificial Sequence
  Description of Artificial Sequence Synthetic primer
tcagtggaggcaggagccaaactgagc

SEQ ID NO: 14 - DNA, 28 bp, Artificial Sequence
  Description of Artificial Sequence Synthetic primer
cacagtccagacacttcttcacactgac

SEQ ID NO: 15 - DNA, 22 bp, Artificial Sequence
  Description of Artificial Sequence Synthetic primer
ctggcagtctatcaacaggtcc

SEQ ID NO: 16 - DNA, 22 bp, Artificial Sequence
  Description of Artificial Sequence Synthetic primer
tgtggagtagagtgaggcttcc

SEQ ID NO: 17 - DNA, 30 bp, Artificial Sequence
  Description of Artificial Sequence Synthetic primer
tatgacggcagcactggcaagttctactgc

SEQ ID NO: 18 - DNA, 30 bp, Artificial Sequence
  Description of Artificial Sequence Synthetic primer
atgggtagttgcagtcagttggtatcatgg

SEQ ID NO: 19 - DNA, 30 bp, Artificial Sequence
  Description of Artificial Sequence Synthetic primer
atgttagagaagtagttccagatatgctgg

SEQ ID NO: 20 - DNA, 30 bp, Artificial Sequence
  Description of Artificial Sequence Synthetic primer
cagtgagacagacaatcatggatggagacg

SEQ ID NO: 21 - DNA, 30 bp, Artificial Sequence
  Description of Artificial Sequence Synthetic primer
ctgcactgccaagactgaatggctggatgg

SEQ ID NO: 22 - DNA, 30 bp, Artificial Sequence
  Description of Artificial Sequence Synthetic primer
ggacgctctcctgagctacagaaggaatgg

SEQ ID NO: 23 - DNA, 28 bp, Artificial Sequence
  Description of Artificial Sequence Synthetic primer
tgcctttgccacaaggaaagtggcaggc

SEQ ID NO: 24 - DNA, 28 bp, Artificial Sequence
  Description of Artificial Sequence Synthetic primer
tccgactgactattgtagtgtttgatgc

SEQ ID NO: 25 - DNA, 30 bp, Artificial Sequence
  Description of Artificial Sequence Synthetic primer
gctctgattcttgccaaactgagactcttc

SEQ ID NO: 26 - DNA, 30 bp, Artificial Sequence
  Description of Artificial Sequence Synthetic primer
aggaagctaagacccactactacatacacc

(19) United States
(12) Patent Application Publication
(10) Pub. No.: US 2014/0328847 A1
(43) Pub. Date: Nov. 6, 2014

(54) MITIGATION OF DISEASE BY INHIBITION OF GALECTIN-12

(71) Applicant: The Regents of the University of California, Oakland, CA (US)

(72) Inventors: Ri-Yao Yang, Davis, CA (US); Fu-Tong Liu, West Sacramento, CA (US); Lan Yu, Woodland, CA (US); Daniel K. Hsu, Davis, CA (US)

(73) Assignee: THE REGENTS OF THE UNIVERSITY OF CALIFORNIA, Oakland, CA (US)

(21) Appl. No.: 14/348,612

(22) PCT Filed: Oct. 2, 2012
(86) PCT No.: PCT/US12/58403

§ 371 Date: Mar. 31, 2014

Related U.S. Application Data
(60) Provisional application No. 61/542,608, filed on Oct. 3, 2011.

(51) Int. Cl. C12N 15/113; C07K 16/28

(52) U.S. Cl. CPC C12N 15/113; C07K 16/2851; C12N 2310/11; C12N 2320/30; C07K 2316/96
USPC 424/137.1; 514/44 A

(57) ABSTRACT

It has now been discovered that mice with an ablated galectin-12 gene exhibit enhanced fat mobilization (lipolysis), have reduced adipose tissue mass, improved insulin sensitivity and glucose tolerance, and increased mitochondrial respiration. Inhibition of galectin-12 activity can therefore be used to reduce, mitigate, inhibit and/or prevent obesity, type 2 diabetes, metabolic diseases, mitochondrial diseases, other disease conditions associated with and/or caused by the abnormal expression or overexpression of galectin-12, and other disease conditions with normal galectin-12 expression but will benefit from galectin-12 inhibition.

### CROSS-REFERENCE TO RELATED APPLICATIONS

This application is a U.S. National Phase filing under 35 U.S.C. §371 of Intl. Application No. PCT/US2012/058403, filed on Oct. 2, 2012, which claims the benefit under 35 U.S.C. §119(e) of U.S. Provisional Application No. 61/542,608, filed on Oct. 3, 2011, both of which are hereby incorporated herein by reference in their entirety for all purposes.

### STATEMENT OF GOVERNMENTAL SUPPORT

This invention was made with government support under Grant Nos. RO1AI020958 and RO1AR056343, awarded by the National Institutes of Health. The government has certain rights in the invention.

### FIELD OF THE INVENTION

The present invention relates to reducing, mitigating, inhibiting and/or preventing disease conditions associated with or caused by the abnormal expression or overexpression of galectin-12, by inhibiting galectin-12 activity. It also applies to disease conditions with normal galectin-12 expression but which will benefit from inhibiting galectin-12 activity.

### BACKGROUND OF THE INVENTION

The galectin family of animal lectins encompasses 15 members in mammals with conserved carbohydrate-recognition domains (CRD) that bind β-galactoside (1). Unlike most other animal lectins that are synthesized on endoplasmic reticulum (ER)-bound ribosomes and delivered to the cell surface or secreted via the ER/Golgi pathway, galectins possess characteristics of intracellular proteins and are synthesized on free ribosomes (2). However, they can also be detected on the cell surface and extracellular space. Previous research describe galectins as possessing both intracellular and extracellular functions in a variety of cellular processes, including cell-cell and cell-extracellular matrix interactions, intracellular vesicle trafficking, cell growth, apoptosis, and cell activation that impact innate and adaptive immunity, as well as cancer initiation, progression, and metastasis (reviewed in (2-7)).

We cloned cDNA coding for a human two-CRD galectin, galectin-12 (8). The mouse gene was subsequently cloned by Hotta et al (9) who also showed by Northern blotting its preferential expression in adipose tissue. Using the serial analysis of gene expression (SAGE) strategy, the gene was later found to be one of the few genes that are specifically expressed in mouse adipose tissue (10). At times of energy surplus, fatty acids are converted into triglycerides in these cells and stored in specialized lipid droplet organelles (11). When needed, triglyceride can be hydrolyzed into fatty acids and glycerol in a tightly controlled process known as lipolysis (12). There is a delicate balance between triglyceride synthesis and lipolysis in healthy animals. Disturbance of such a balance can result in lipodystrophy or obesity. It is well established that both obesity and lipodystrophy can result in insulin resistance, type 2 diabetes, and an increased risk for cardiovascular disease (13).

Lipolysis is regulated by opposing mechanisms, largely via modulation of intracellular concentrations of cAMP. In the present studies, we have investigated the localization of galectin-12 and the role of this protein in adipocytes and adiposity by generating and studying galectin-12-deficient (Lgals12^{-/-}) mice. Here we demonstrate that galectin-12 is a lipid droplet protein that regulates lipolytic PKA signaling. Galectin-12 deficiency in mice results in enhanced lipolysis, reduced adiposity, and ameliorated insulin resistance.

### SUMMARY OF THE INVENTION

In one aspect, the invention provides methods of promoting lipolysis and/or reducing adiposity in a subject. In some embodiments, the methods comprises administering to the subject an effective amount of an inhibitor of galectin-12 activity, thereby promoting lipolysis and/or reducing adiposity in the subject.

In another aspect, the invention provides methods of promoting and/or increasing insulin sensitivity and/or glucose tolerance in a subject. In some embodiments, the methods comprise administering to the subject an effective amount of an inhibitor of galectin-12 activity, thereby promoting and/or increasing insulin sensitivity and/or glucose tolerance in the subject.

In some embodiments, the subject is obese. In some embodiments, the subject is overweight. In some embodiments, the subject has type 2 diabetes. In some embodiments, the subject is a pre-diabetic, e.g., with hyperglycemia and/or insulin insensitivity. In some embodiments, the subject has metabolic disease. In some embodiments, the subject has cardiovascular disease. In some embodiments, the subject has renal disease.

In a further aspect, the invention provides methods of preventing, inhibiting, mitigating, or delaying one or more symptoms of a mitochondrial disease in a subject. In some embodiments, the methods comprise administering to the subject an effective amount of an inhibitor of galectin-12 activity, thereby preventing, inhibiting, mitigating, or delaying one or more symptoms of the mitochondrial disease in the subject.

In another aspect, the invention provides methods of promoting and/or increasing mitochondrial respiration in a subject. In some embodiments, the methods comprise administering to the subject an effective amount of an inhibitor of galectin-12 activity, thereby promoting and/or increasing mitochondrial respiration in the subject.

In some embodiments, the subject has a mitochondrial disease resulting from dysfunctional mitochondria in cells wherein galectin-12 is constitutively expressed or abnormally overexpressed. In some embodiments, the subject has a mitochondrial disease selected from the group consisting of Luft disease, Leigh syndrome (Complex I, cytochrome oxidase (COX) deficiency, pyruvate dehydrogenase (PDH) deficiency), Alpers Disease, Medium-Chain Acyl-CoA Dehydrongenase Deficiency (MCAD), Short-Chain Acyl-CoA Dehydrogenase Deficiency (SCAD), Short-chain-3-hydroxyacyl-CoA dehydrogenase (SCHAD) deficiency, Very Long-Chain Acyl-CoA Dehydrongenase Deficiency (VLCAD), Long-chain 3-hydroxyacyl-CoA dehydrogenase (LCHAD) deficiency, glutaric aciduria II, lethal infantile cardiomyopathy, Friedreich ataxia, maturity onset diabetes of young, malignant hyperthermia, disorders of ketone utilization, mtDNA depletion syndrome, reversible cox deficiency of infancy, various defects of the Krebs cycle, pyruvate dehydrogenase deficiency, pyruvate carboxylase deficiency, fumarase deficiency, carnitine palmitoyl transferase deficiency.

In some embodiments, the subject has a cancer associated with or caused by the constitutive expression or overexpression of galectin-12. In some embodiments, the cancer is selected from the group consisting of acute myeloid leukemia type M3 (acute promyelocytic leukemia), melanoma, and neuroblastoma.

In some embodiments, the inhibitor of galectin-12 activity inhibits the binding of galectin-12 to beta-galactose-containing ligands or its proteinaceous binding partners. In some embodiments, the inhibitor of galectin-12 activity inhibits the binding of galectin-12 to beta-galactosides, or galactoside-containing glycans. In some embodiments, the inhibitor of galectin 12 activity inhibits the binding of galectin-12 to one or more proteinaceous binding partners selected from the group consisting of mitochondrial chaperone HSP60, heat-shock cognate 70 (Hsc70), and vacuolar protein sorting 13 (VPS13). In some embodiments, the inhibitor of galectin-12 activity is a glycan mimetic. In some embodiments, the inhibitor of galectin-12 activity is a peptide. In some embodiments, the inhibitor of galectin-12 is an antigen binding molecule. In some embodiments, the inhibitor of galectin-12 is an antibody or fragment thereof. In some embodiments, the inhibitor of galectin-12 is a nucleic acid (e.g., DNA or RNA aptamer).

In some embodiments, the inhibitor of galectin-12 activity is identified in a library of compounds having a core structure as depicted in FIG. 14, FIG. 15A, FIG. 15B, FIG. 16, or FIG. 17. In some embodiments, the inhibitor of galectin-12 activity comprises a substituted core comprised of a galactose, lactose, an oligo-lactose, a poly-lactose, thiodigalactose, or analogs and/or derivatives thereof. In some embodiments, the inhibitor of galectin-12 activity comprises a galactose, a lactose, an oligo-lactose, a poly-lactose or a thiodigalactose nucleus attached to a scaffold comprising one or more linear, cyclic, aromatic, polycyclic linkers, wherein a library of functional groups is connected to the one or more linkers. In some embodiments, the inhibitor of galectin-12 activity comprises a N-Acetyl-lactosamine core. In some embodiments, the inhibitor of galectin-12 activity comprises a thiodigalactose core. In some embodiments, the inhibitor of galectin-12 activity is administered orally, intravenously, topically, transdermally, or delivered in situ to the therapeutic location (e.g., delivered directly to the required site of action in the body). In some embodiments, the inhibitor of galectin-12 activity is encapsulated in a liposome or micellar nanocarrier.

In some embodiments, the galectin-12 polypeptide that is inhibited is selected from a galectin-12 isoform 1, a galectin-12 isoform 2, a galectin-12 isoform 3, a galectin-12 isoform 4, a galectin-12 isoform 5. In some embodiments, the galectin-12 polypeptide that is inhibited has at least 90%, 91%, 92%, 93%, 94%, 95%, 95%, 96%, 97%, 98%, 99% or 100% sequence identity to NP_{—}001136007.1 (isoform 1); NP_{—}149092.2 (isoform 2); NP_{—}001136008.1 (isoform 3); NP_{—}001136009.1 (isoform 4); or NP_{—}001136010.1 (isoform 5).

In some embodiments, the inhibitor of galectin 12 activity inhibits the expression of galectin-12. In some embodiments, the inhibitor of galectin-12 expression binds to or hybridizes to the galectin-12 promoter. In some embodiments, the inhibitor of galectin 12 expression is an inhibitory nucleic acid. In some embodiments, the inhibitor of galectin 12 expression is small interfering RNA (siRNA). In some embodiments, the inhibitor of galectin 12 expression is an inhibitory nucleic acid that hybridizes to a nucleic acid sequence encoding galectin-12 and having at least 90%, 91%, 92%, 93%, 94%, 95%, 95%, 96%, 97%, 98%, 99% or 100% sequence identity to NM_{—}001142535.1 (isoform 1); NM_{—}033101.3 (isoform 2); NM_{—}001142536.1 (isoform 3); NM_{—}001142537.1 (isoform 4); or NM_{—}001142538.1 (isoform 5).

In some embodiments, the subject is a human. In some embodiments, the subject is a domesticated mammal. In some embodiments, the subject is an agricultural mammal.

### DEFINITIONS

The following words and phrases are intended to have the meanings as set forth below, except to the extent that the context in which they are used indicates otherwise or they are expressly defined to mean something different.

The term “galectins” (also known as galaptins or S-lectin) refers to a family of animal lectins with conserved carbohydrate-recognition domains (CRDs) for β-galactoside (Barondes, S. H. et al. J. Biol. Chem. 269:20807-20810 (1994)). They are present in most species of the animal kingdom, including lower organisms, such as nematodes, and higher organisms, such as mammals. In mammals, fifteen members have been identified and more are likely to be discovered as more genomes are sequenced (Cooper, D. N. Biochim Biophys Acta. 1572:209-231 (2002); Cummings R D, Liu F (2009) in Essentials of Glycobiology, eds Varki A, Cummings R D, Esko J D, Freeze H H, Stanley P, Bertozzi C R, Hart G W, Etzler M E (Cold Spring Harbor Laboratory Press, Cold Spring Harbor, N.Y.), pp 475-488). The family can be subdivided into prototypical type (galectin-1, -2, -5, -7, -10, -13, -14 and 15), which are monomers or homodimers of one carbohydrate-recognition domain (.about.15 kDa); tandem repeat type (galectin-4, -6, -8, -9, and -12), which contain two distinct but homologous CRD in a single polypeptide chain; and chimeric type, where galectin-3 is the only member and contains a non-lectin part made of proline-, glycine-rich short tandem repeats connected to a CRD. Some of the members, especially galectin-3, which was first cloned from rat basophilic leukemic cells by this group by virtue of its binding to IgE (Liu, F. T. et al. Proc Natl Acad Sci USA. 82:4100-4104 (1985)), and galectin-1, have been extensively studied, and experimental results suggest that these lectins may have diverse functions (Liu, F. T. Clin Immunol. 97:79-88 (2000); Liu, F. T. et al. Biochim Biophys Acta. 1572:263-273 (2002)).

Most galectins have wide tissue distribution. Galectin-3, for example, is abundantly present in the epithelia of several organs (Liu, F. T. Clin Immunol. 97:79-88 (2000)), as well as in various inflammatory cells, including monocytes/macrophages (Liu, F. T. et al. Am J. Pathol. 147:1016-1028 (1995)). Consistent with the lack of a classical signal sequence, galectins are mainly intracellular proteins (Liu, F. T. Clin Immunol. 97:79-88 (2000)). However, a number of studies have demonstrated the secretion of these proteins. The mechanism underlying the secretion of galectins is not well understood, but plasma membrane targeting and vesicular budding are thought to be critically involved in the secretion of galectin-3 (Mehul, B. et al. J Cell Sci. 110(10):1169-1178 (1997)). More recently, galectin-3 has been identified as a component of exosomes in dendritic cells (Thery, C. et al., J. Immunol. 166:7309-7318 (2001)), suggesting an interesting possibility that this lectin and other galectins are secreted as a part of exosomes. Consistent with their spatial distribution, these proteins appear to function both intracellularly and extracellularly. The extracellular functions are likely to be due to carbohydrate-binding properties and in many cases are inhibited by specific free carbohydrate, while the intracellular functions may not be related to carbohydrate binding (Liu, F. T. et al. Biochim Biophys Acta. 1572:263-273 (2002)). Although all galectins contain at least one homologous CRD in their sequence, different members exhibit different localization and expression patterns, suggesting distinct functions for each member of the family (Lowe, J. B. Cell, 104:809-812 (2001); Rabinovich, G. A. et al., Trends Immunol. 23:313-320 (2002); Yang, R. Y. et al. Cell Mol Life Sci. 60:267-276 (2003)).

Galectin-12 refers to a galectin with two CRDs. The N-terminal CRD is highly homologous to those of other galectins, while its C-terminal CRD shows significant divergence (Yang, R. Y. et al., J. Biol. Chem. 276:20252-20260 (2001) (hereafter, “Yang 2001”)). Its mRNA contains AU-rich motifs in the 3′-untranslated region, and the initiation codon for translation locates in a suboptimal context (Yang 2001), suggesting vigorous post-transcriptional regulation at the levels of mRNA stability (Chen, C. Y. et al., Trends Biochem Sci. 20:465-470 (1995)) and translation efficiency (Kozak, M. Gene, 299:1-34 (2002)). The expression of this gene is very restricted, with high expression only in adipocytes (Hotta, K. et al. J. Biol. Chem. 276:34089-34097 (2001) (hereafter, “Hotta 2001”)) and peripheral blood leucocytes (Yang 2001). Galectin-12 is up-regulated when cells are blocked at the G1 phase and ectopic expression of this protein causes cell cycle arrest at the G1 phase with concomitant cell growth suppression (Yang 2001). Its expression in adipocytes is down-regulated by agents known to impair insulin sensitivity, implying a role for galectin-12 in the pathogenesis of type 2 diabetes (Fasshauer, M. et al. Eur J. Endocrinol. 147:553-559 (2002)). Galectin-12 has been sequenced, as reported by Yang, R. Y. et al. J. Biol. Chem. 276:20252-20260 (2001) and Strausberg et al. PNAS 99(26):16899-16903 (2002). Nucleic Acid and amino acid sequences of five different isoforms of Galectin-12 are known, e.g., GenBank Accession Nos. NM_{—}001142535.1→NP_{—}001136007.1 (isoform 1); NM_{—}033101.3→NP 149092.2 (isoform 2); NM_{—}001142536.1→NP_{—}001136008.1 (isoform 3); NM_{—}001142537.1→NP_{—}001136009.1 (isoform 4); and NM_{—}001142538.1→NP_{—}001136010.1 (isoform 5).

The phrase “sequence identity,” in the context of two nucleic acids or polypeptides, refers to two or more sequences or subsequences that have a certain level of nucleotide or amino acid residue identity, when compared and aligned for maximum correspondence, as measured using one of the following sequence comparison algorithms or by visual inspection. Preferably, the aligned sequences share at least 90% sequence identity, for example, at least 95%, 96%, 97%, 98%, 99% or 100% sequence identity to a reference sequence (e.g., GenBank Accession Nos. NM_{—}001142535.1→NP_{—}001136007.1 (isoform 1); NM_{—}033101.3→NP_{—}149092.2 (isoform 2); NM_{—}001142536.1→NP_{—}001136008.1 (isoform 3); NM_{—}001142537.1→NP_{—}001136009.1 (isoform 4); and NM_{—}001142538.1→NP_{—}001136010.1 (isoform 5)). The sequence identity can exist over a region of the sequences that is at least about 10, 20 or 50 residues in length, sometimes over a region of at least about 100 or 150 residues. In some embodiments, the sequences share a certain level of sequence identity over the entire length of the sequence of interest.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (on the World Wide Web at ncbi.nhn.nih.gov/) (see Henikoff & Henikoff, Proc. Natl. Acad. Sci. USA 89:10915 (1989)).

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” as used herein applies to amino acid sequences. One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins (1984)).

The terms “decrease” or “reduce” or “inhibit” interchangeably refer to the detectable reduction of a measured response (e.g., galectin-12 binding and/or expression). The decrease, reduction or inhibition can be partial, for example, at least 10%, 25%, 50%, 75%, or can be complete (i.e., 100%). The decrease, reduction or inhibition can be measured in comparison to a control. For example, decreased, reduced or inhibited responses can be compared before and after treatment. Decreased, reduced or inhibited responses can also be compared to an untreated control, or to a known value.

The term “binding partner analog” refers to a compound (e.g., small molecules) that shares structural and/or functional similarity with at least a part of a binding partner (e.g., a fragment of a binding partner), but unlike the endogenous binding partner, the binding partner analog inhibits the function of the protein (i.e., galectin-12) upon binding. A binding partner analog can have structural similarity to at least a part of a binding partner as measured on a 2-dimensional or 3-dimensional (electron densities, location of charged, uncharged and/or hydrophobic moieties) basis. A binding partner analog can have functional similarity with at least a part of a binding partner inasmuch as the binding partner analog binds to the protein (i.e., galectin-12). A binding partner analog can be a competitive inhibitor.

A “compound that inhibits galectin-12 activity” refers to any compound that inhibits galectin-12 activity. The inhibition can be, for example, on the transcriptional, translational or protein level. Accordingly, the compound can be in any chemical form, including nucleic acid or nucleotide, amino acid or polypeptide, monosaccharide or oligosaccharide, nucleotide sugar, or small organic molecule.

As used herein, “administering” refers to local and systemic administration, e.g., including enteral and parenteral administration. Routes of administration for the compounds described herein include, e.g., oral (“po”) administration, administration as a suppository, topical contact, intravenous (“iv”), intraperitoneal (“ip”), intramuscular (“im”), intralesional, intranasal, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (i.e., other than through the alimentary tract, such as intramuscular, intravenous, intra-arterial, transdermal and subcutaneous) administration.

The term “co-administering” or “concurrent administration”, when used, for example with respect to the polyphenol compounds described herein and another active agent, refers to administration of a polyphenol compound described and a second active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the body (e.g. in the plasma) at a significant fraction (e.g. 20% or greater, preferably 30% or 40% or greater, more preferably 50% or 60% or greater, most preferably 70% or 80% or 90% or greater) of their maximum serum concentration for any given dose.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies (e.g., disease conditions associated with or caused by the abnormal or aberrant expression or overexpression of galectin-12), or one or more symptoms of such disease or condition. It also applies to diseases or conditions with normal galectin-12 expression but which will benefit from the treatment.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than a polyphenol compound, as described herein.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other healthworker in a hospital, psychiatric care facility, as an outpatient, or other clinical context. In certain embodiments the subject may not be under the care or prescription of a physician or other healthworker.

The term “effective amount” or “therapeutically effective amount” refers to the amount of an active agent sufficient to induce a desired biological result (e.g., prevention, delay, reduction or inhibition of ischemia or symptoms associated with ischemia). That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

“Subtherapeutic dose” refers to a dose of a pharmacologically active agent(s), either as an administered dose of pharmacologically active agent, or actual level of pharmacologically active agent in a subject that functionally is insufficient to elicit the intended pharmacological effect in itself (e.g., to dissolve an embolic clot), or that quantitatively is less than the established therapeutic dose for that particular pharmacological agent (e.g., as published in a reference consulted by a person of skill, for example, doses for a pharmacological agent published in the Physicians' Desk Reference, 66th Ed., 2011, Thomson Healthcare or Brunton, et al., Goodman & Gilman's The Pharmacological Basis of Therapeutics, 11th edition, 2006, McGraw-Hill Professional). A “subtherapeutic dose” can be defined in relative terms (i.e., as a percentage amount (less than 100%) of the amount of pharmacologically active agent conventionally administered). For example, a subtherapeutic dose amount can be about 1% to about 75% of the amount of pharmacologically active agent conventionally administered. In some embodiments, a subtherapeutic dose can be about 75%, 50%, 30%, 25%, 20%, 10% or less, than the amount of pharmacologically active agent conventionally administered.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “obese” or “obesity” refers to an individual who has a body mass index (BMI) of 30 kg/m² or more due to excess adipose tissue. Obesity also can be defined on the basis of body fat content: greater than 25% body fat content for a male or more than 30% body fat content for a female. A “morbidly obese” individual has a body mass index greater than 35 kg/m².

The term “overweight” refers to an individual who has a body mass index of 25 kg/m² or more, but less than 30 kg/m².

The term “body mass index” or “BMI” refers to a weight to height ratio measurement that estimates whether an individual's weight is appropriate for their height. As used herein, an individual's body mass index is calculated as follows: BMI=(pounds×700)/(height in inches)² or BMI=(kilograms)/(height in meters)².

The term “baseline body weight” refers to the body weight presented by the individual at the initiation of treatment.

The terms “prediabetes” and “prediabetic” interchangeably refer to a condition that involves impaired glucose tolerance (IGT) or impaired fasting glucose (IFG). IGT is defined by a 2-h oral glucose tolerance test plasma glucose concentration >140 mg/dL (7.8 mmol/L) but <200 mg/dL (11.1 mmol/L), and IFG is defined by a fasting plasma glucose concentration ≧100 mg/dL (5.6 mmol/L), but <126 mg/dL (7.0 mmol/L). See, e.g., Pour and Dagogo-Jack, Clin Chem. (2010) November 9., PMID 21062906.

The symbol “—” means a single bond, “═” means a double bond, “≡” means a triple bond. The symbol “ [symbol] ” refers to a group on a double-bond as occupying either position on the terminus of the double bond to which the symbol is attached; that is, the geometry, E- or Z-, of the double bond is ambiguous and both isomers are meant to be included. When a group is depicted removed from its parent formula, the “ [symbol] ” symbol will be used at the end of the bond which was theoretically cleaved in order to separate the group from its parent structural formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, CH₂CH₂. It would be understood by one of ordinary skill in the art that the aforementioned descriptive techniques are common in the chemical arts to provide brevity and simplicity to description of otherwise complex structures.

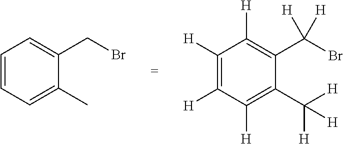

In this application, some ring structures are depicted generically and will be described textually. For example, in the schematic below if ring A is used to describe a phenyl, there are at most four hydrogens on ring A (when R is not H).

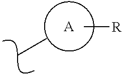

If a group R is depicted as “floating” on a ring system, as for example in the group:

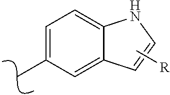

then, unless otherwise defined, a substituent R can reside on any atom of the fused bicyclic ring system, excluding the atom carrying the bond with the “ ” symbol, so long as a stable structure is formed. In the example depicted, the R group can reside on an atom in either the 5-membered or the 6-membered ring of the indolyl ring system.

When there are more than one such depicted “floating” groups, as for example in the formulae:

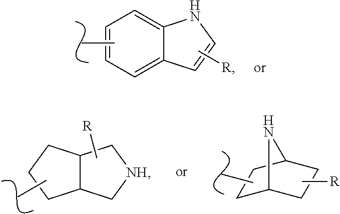

where there are two groups, namely, the R and the bond indicating attachment to a parent structure; then, unless otherwise defined, the “floating” groups can reside on any atoms of the ring system, again assuming each replaces a depicted, implied, or expressly defined hydrogen on the ring system and a chemically stable compound would be formed by such an arrangement.

When a group R is depicted as existing on a ring system containing saturated carbons, as for example in the formula:

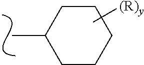

where, in this example, y can be more than one, assuming each replaces a currently depicted, implied, or expressly defined hydrogen on the ring; then, unless otherwise defined, two R's can reside on the same carbon. A simple example is when R is a methyl group; there can exist a geminal dimethyl on a carbon of the depicted ring (an “annular” carbon). In another example, two R's on the same carbon, including that same carbon, can form a ring, thus creating a spirocyclic ring (a “spirocyclyl” group) structure. Using the previous example, where two R's form, e.g. a piperidine ring in a spirocyclic arrangement with the cyclohexane, as for example in the formula:

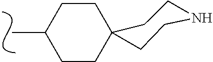

“Alkyl” in its broadest sense is intended to include linear, branched, or cyclic hydrocarbon structures, and combinations thereof. Alkyl groups can be fully saturated or with one or more units of unsaturation, but not aromatic. Generally alkyl groups are defined by a subscript, either a fixed integer or a range of integers. For example, “C₈alkyl” includes n-octyl, iso-octyl, 3-octynyl, cyclohexenylethyl, cyclohexylethyl, and the like; where the subscript “8” designates that all groups defined by this term have a fixed carbon number of eight. In another example, the term “C₁₋₆alkyl” refers to alkyl groups having from one to six carbon atoms and, depending on any unsaturation, branches and/or rings, the requisite number of hydrogens. Examples of C₁₋₆alkyl groups include methyl, ethyl, vinyl, propyl, isopropyl, butyl, s-butyl, t-butyl, isobutyl, isobutenyl, pentyl, pentynyl, hexyl, cyclohexyl, hexenyl, and the like. When an alkyl residue having a specific number of carbons is named generically, all geometric isomers having that number of carbons are intended to be encompassed. For example, either “propyl” or “C₃alkyl” each include n-propyl, c-propyl, propenyl, propynyl, and isopropyl. Cycloalkyl is a subset of alkyl and includes cyclic hydrocarbon groups of from three to thirteen carbon atoms. Examples of cycloalkyl groups include c-propyl, c-butyl, c-pentyl, norbornyl, norbornenyl, c-hexenyl, adamantyl and the like. As mentioned, alkyl refers to alkanyl, alkenyl, and alkynyl residues (and combinations thereof)—it is intended to include, e.g., cyclohexylmethyl, vinyl, allyl, isoprenyl, and the like. An alkyl with a particular number of carbons can be named using a more specific but still generic geometrical constraint, e.g. “C₃₋₆cycloalkyl” which means only cycloalkyls having between 3 and 6 carbons are meant to be included in that particular definition. Unless specified otherwise, alkyl groups, whether alone or part of another group, e.g. —C(O)alkyl, have from one to twenty carbons, that is C₁₋₂₀alkyl. In the example “—C(O)alkyl,” where there were no carbon count limitations defined, the carbonyl of the —C(O)alkyl group is not included in the carbon count, since “alkyl” is designated generically. But where a specific carbon limitation is given, e.g. in the term “optionally substituted C₁₋₂₀alkyl,” where the optional substitution includes “oxo” the carbon of any carbonyls formed by such “oxo” substitution are included in the carbon count since they were part of the original carbon count limitation. However, again referring to “optionally substituted C₁₋₂₀alkyl,” if optional substitution includes carbon-containing groups, e.g. CH₂CO₂H, the two carbons in this group are not included in the C₁₋₂₀alkyl carbon limitation.

When a carbon number limit is given at the beginning of a term which itself comprises two terms, the carbon number limitation is understood as inclusive for both terms. For example, for the term “C₇₋₁₄arylalkyl,” both the “aryl” and the “alkyl” portions of the term are included the carbon count, a maximum of 14 in this example, but additional substituent groups thereon are not included in the atom count unless they incorporate a carbon from the group's designated carbon count, as in the “oxo” example above. Likewise when an atom number limit is given, for example “6-14 membered heteroarylalkyl,” both the “heteroaryl” and the “alkyl” portion are included the atom count limitation, but additional substituent groups thereon are not included in the atom count unless they incorporate a carbon from the group's designated carbon count. In another example, “C₄₋₁₀cycloalkylalkyl” means a cycloalkyl bonded to the parent structure via an alkylene, alkylidene or alkylidyne; in this example the group is limited to 10 carbons inclusive of the alkylene, alkylidene or alkylidyne subunit. As another example, the “alkyl” portion of, e.g. “C₇₋₁₄arylalkyl” is meant to include alkylene, alkylidene or alkylidyne, unless stated otherwise, e.g. as in the terms “C₇₋₁₄arylalkylene” or “C₆₋₁₀aryl-CH₂CH₂—.”

“Alkylene” refers to straight, branched and cyclic (and combinations thereof) divalent radical consisting solely of carbon and hydrogen atoms, containing no unsaturation and having from one to ten carbon atoms, for example, methylene, ethylene, propylene, n-butylene and the like. Alkylene is like alkyl, referring to the same residues as alkyl, but having two points of attachment and, specifically, fully saturated. Examples of alkylene include ethylene (—CH₂CH₂—), propylene (—CH₂CH₂CH₂—), dimethylpropylene (—CH₂C(CH₃)₂CH₂—), cyclohexan-1,4-diyl and the like.

“Alkylidene” refers to straight, branched and cyclic (and combinations thereof) unsaturated divalent radical consisting solely of carbon and hydrogen atoms, having from two to ten carbon atoms, for example, ethylidene, propylidene, n-butylidene, and the like. Alkylidene is like alkyl, referring to the same residues as alkyl, but having two points of attachment and, specifically, at least one unit of double bond unsaturation. Examples of alkylidene include vinylidene (—CH═CH—), cyclohexylvinylidene (—CH═C(C₆H₁₃)—), cyclohexen-1,4-diyl and the like.

“Alkylidyne” refers to straight, branched and cyclic (and combinations thereof) unsaturated divalent radical consisting solely of carbon and hydrogen atoms having from two to ten carbon atoms, for example, propylid-2-ynyl, n-butylid-1-ynyl, and the like. Alkylidyne is like alkyl, referring to the same residues as alkyl, but having two points of attachment and, specifically, at least one unit of triple bond unsaturation.

Any of the above radicals” “alkylene,” “alkylidene” and “alkylidyne,” when optionally substituted, can contain alkyl substitution which itself can contain unsaturation. For example, 2-(2-phenylethynyl-but-3-enyl)-naphthalene (IUPAC name) contains an n-butylid-3-ynyl radical with a vinyl substituent at the 2-position of the radical. Combinations of alkyls and carbon-containing substitutions thereon are limited to thirty carbon atoms.

“Alkoxy” refers to the group —O-alkyl, where alkyl is as defined herein. Alkoxy includes, by way of example, methoxy, ethoxy, n-propoxy, isopropoxy, n-butoxy, t-butoxy, sec-butoxy, n-pentoxy, cyclohexyloxy, cyclohexenyloxy, cyclopropylmethyloxy, and the like.

“Haloalkyloxy” refers to the group —O-alkyl, where alkyl is as defined herein, and further, alkyl is substituted with one or more halogens. By way of example, a haloC₁₋₃alkyloxy” group includes —OCF₃, —OCF₂H, —OCHF₂, —OCH₂CH₂Br, —OCH₂CH₂CH₂I, —OC(CH₃)₂Br, —OCH₂Cl and the like.

“Acyl” refers to the groups —C(O)H, —C(O)alkyl, —C(O)aryl and C(O)heterocyclyl.

“α-Amino Acids” refer to naturally occurring and commercially available α-amino acids and optical isomers thereof. Typical natural and commercially available α-amino acids are glycine, alanine, serine, homoserine, threonine, valine, norvaline, leucine, isoleucine, norleucine, aspartic acid, glutamic acid, lysine, ornithine, histidine, arginine, cysteine, homocysteine, methionine, phenylalanine, homophenylalanine, phenylglycine, ortho-tyrosine, meta-tyrosine, para-tyrosine, tryptophan, glutamine, asparagine, proline and hydroxyproline. A “side chain of an α-amino acid” refers to the radical found on the α-carbon of an α-amino acid as defined above, for example, hydrogen (for glycine), methyl (for alanine), benzyl (for phenylalanine), etc.

“Amino” refers to the group NH₂.

“Amide” refers to the group C(O)NH₂ or —N(H)acyl.

“Aryl” (sometimes referred to as “Ar”) refers to a monovalent aromatic carbocyclic group of, unless specified otherwise, from 6 to 15 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, 9,10-dihydrophenanthrenyl, indanyl, tetralinyl, and fluorenyl and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic or tetracyclic.

“Arylene” refers to an aryl that has at least two groups attached thereto. For a more specific example, “phenylene” refers to a divalent phenyl ring radical. A phenylene, thus can have more than two groups attached, but is defined by a minimum of two non-hydrogen groups attached thereto.

“Arylalkyl” refers to a residue in which an aryl moiety is attached to a parent structure via one of an alkylene, alkylidene, or alkylidyne radical. Examples include benzyl, phenethyl, phenylvinyl, phenylallyl and the like. When specified as “optionally substituted,” both the aryl, and the corresponding alkylene, alkylidene, or alkylidyne portion of an arylalkyl group can be optionally substituted. By way of example, “C₇₋₁₁arylalkyl” refers to an arylalkyl limited to a total of eleven carbons, e.g., a phenylethyl, a phenylvinyl, a phenylpentyl and a naphthylmethyl are all examples of a “C₇₋₁₁arylalkyl” group.

“Aryloxy” refers to the group —O-aryl, where aryl is as defined herein, including, by way of example, phenoxy, naphthoxy, and the like.

“Carboxyl,” “carboxy” or “carboxylate” refers to CO₂H or salts thereof.

“Carboxyl ester” or “carboxy ester” or “ester” refers to the group —CO₂alkyl, —CO₂aryl or —CO₂heterocyclyl.

“Carbonate” refers to the group —OCO₂alkyl, —OCO₂aryl or —OCO₂heterocyclyl.

“Carbamate” refers to the group —OC(O)NH₂, —N(H)carboxyl or —N(H)carboxyl ester.

“Cyano” or “nitrile” refers to the group —CN.

“Formyl” refers to the specific acyl group —C(O)H.

“Halo” or “halogen” refers to fluoro, chloro, bromo and iodo.

“Haloalkyl” and “haloaryl” refer generically to alkyl and aryl radicals that are substituted with one or more halogens, respectively. By way of example “dihaloaryl,” “dihaloalkyl,” “trihaloaryl” etc. refer to aryl and alkyl substituted with a plurality of halogens, but not necessarily a plurality of the same halogen; thus 4-chloro-3-fluorophenyl is a dihaloaryl group.

“Heteroalkyl” refers to an alkyl where one or more, but not all, carbons are replaced with a heteroatom. A heteroalkyl group has either linear or branched geometry. By way of example, a “2-6 membered heteroalkyl” is a group that can contain no more than 5 carbon atoms, because at least one of the maximum 6 atoms must be a heteroatom, and the group is linear or branched. Also, for the purposes of this invention, a heteroalkyl group always starts with a carbon atom, that is, although a heteroalkyl may contain one or more heteroatoms, the point of attachment to the parent molecule is not a heteroatom. A 2-6 membered heteroalkyl group includes, for example, —CH₂XCH₃, —CH₂CH₂XCH₃, —CH₂CH₂XCH₂CH₃, C(CH₂)₂XCH₂CH₃ and the like, where X is O, NH, NC₁₋₆alkyl and S(O)₀₋₂, for example.

“Perhalo” as a modifier means that the group so modified has all its available hydrogens replaced with halogens. An example would be “perhaloalkyl.” Perhaloalkyls include —CF₃, —CF₂CF₃, perchloroethyl and the like.

“Hydroxy” or “hydroxyl” refers to the group —OH.

“Heteroatom” refers to O, S, N, or P.

“Heterocyclyl” in the broadest sense includes aromatic and non-aromatic ring systems and more specifically refers to a stable three- to fifteen-membered ring radical that consists of carbon atoms and from one to five heteroatoms. For purposes of this description, the heterocyclyl radical can be a monocyclic, bicyclic or tricyclic ring system, which can include fused or bridged ring systems as well as spirocyclic systems; and the nitrogen, phosphorus, carbon or sulfur atoms in the heterocyclyl radical can be optionally oxidized to various oxidation states. In a specific example, the group —S(O)₀₋₂—, refers to —S— (sulfide), —S(O)— (sulfoxide), and —SO₂— (sulfone) linkages. For convenience, nitrogens, particularly but not exclusively, those defined as annular aromatic nitrogens, are meant to include their corresponding N-oxide form, although not explicitly defined as such in a particular example. Thus, for a compound having, for example, a pyridyl ring; the corresponding pyridyl-N-oxide is meant to be included in the presently disclosed compounds. In addition, annular nitrogen atoms can be optionally quaternized. “Heterocycle” includes heteroaryl and heteroalicyclyl, that is a heterocyclic ring can be partially or fully saturated or aromatic. Thus a term such as “heterocyclylalkyl” includes heteroalicyclylalkyls and heteroarylalkyls. Examples of heterocyclyl radicals include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, diazabicycloheptane, diazapane, diazepine, tetrahydrofuryl, tetrahydropyranyl, thienyl, benzothieliyl, thiamorpholinyl, thiamorpholinyl sulfoxide, thiamorpholinyl sulfone, dioxaphospholanyl, and oxadiazolyl.

“Heteroaryl” refers to an aromatic group having from 1 to 10 annular carbon atoms and 1 to 4 annular heteroatoms. Heteroaryl groups have at least one aromatic ring component, but heteroaryls can be fully unsaturated or partially unsaturated. If any aromatic ring in the group has a heteroatom, then the group is a heteroaryl, even, for example, if other aromatic rings in the group have no heteroatoms. For example, 2H-pyrido[3,2-b][1,4]oxazin-3(4H)-one-7-yl, indolyl and benzimidazolyl are “heteroaryls.” Heteroaryl groups can have a single ring (e.g., pyridinyl, imidazolyl or furyl) or multiple condensed rings (e.g., indolizinyl, quinolinyl, benzimidazolyl or benzothienyl), where the condensed rings may or may not be aromatic and/or contain a heteroatom, provided that the point of attachment to the parent molecule is through an atom of the aromatic portion of the heteroaryl group. In one embodiment, the nitrogen and/or sulfur ring atom(s) of the heteroaryl group are optionally oxidized to provide for the N-oxide (N→O), sulfinyl, or sulfonyl moieties. Compounds described herein containing phosphorous, in a heterocyclic ring or not, include the oxidized forms of phosphorous. Heteroaryl groups are monocyclic, bicyclic, tricyclic or tetracyclic.

“Heteroaryloxy” refers to O-heteroaryl.

“Heteroarylene” generically refers to any heteroaryl that has at least two groups attached thereto. For a more specific example, “pyridylene” refers to a divalent pyridyl ring radical. A pyridylene, thus can have more than two groups attached, but is defined by a minimum of two non-hydrogen groups attached thereto.

“Heteroalicyclic” refers specifically to a non-aromatic heterocyclyl radical. A heteroalicyclic may contain unsaturation, but is not aromatic. As mentioned, aryls and heteroaryls are attached to the parent structure via an aromatic ring. So, e.g., 2H-1,4-benzoxazin-3(4H)-one-4-yl is a heteroalicyclic, while 2H-1,4-benzoxazin-3(4H)-one-7-yl is an aryl. In another example, 2H-pyrido[3,2-b][1,4]oxazin-3(4H)-one-4-yl is a heteroalicyclic, while 2H-pyrido[3,2-b][1,4]oxazin-3(4H)-one-7-yl is a heteroaryl.

“Heterocyclylalkyl” refers to a heterocyclyl group linked to the parent structure via e.g an alkylene linker, for example (tetrahydrofuran-3-yl)methyl- or (pyridin-4-yl)methyl

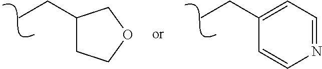

“Heterocyclyloxy” refers to the group —O-heterocycyl.

“Nitro” refers to the group —NO₂.

“Oxo” refers to a double bond oxygen radical, ═O.

“Oxy” refers to —O.radical (also designated as →O), that is, a single bond oxygen radical. By way of example, N-oxides are nitrogens bearing an oxy radical.

When a group with its bonding structure is denoted as being bonded to two partners; that is, a divalent radical, for example, —OCH₂—, then it is understood that either of the two partners can be bound to the particular group at one end, and the other partner is necessarily bound to the other end of the divalent group, unless stated explicitly otherwise. Stated another way, divalent radicals are not to be construed as limited to the depicted orientation, for example “—OCH₂-” is meant to mean not only “—OCH₂—” as drawn, but also “—CH₂O—.”

“Optional” or “optionally” means that the subsequently described event or circumstance may or may not occur, and that the description includes instances where said event or circumstance occurs and instances in which it does not. One of ordinary skill in the art would understand that, with respect to any molecule described as containing one or more optional substituents, that only synthetically feasible compounds are meant to be included. “Optionally substituted” refers to all subsequent modifiers in a term, for example in the term “optionally substituted arylC₁₋₈alkyl,” optional substitution may occur on both the “C₁₋₈alkyl” portion and the “aryl” portion of the arylC₁₋₈alkyl group. Also by way of example, optionally substituted alkyl includes optionally substituted cycloalkyl groups. The term “substituted,” when used to modify a specified group or radical, means that one or more hydrogen atoms of the specified group or radical are each, independently of one another, replaced with the same or different substituent groups as defined below. Thus, when a group is defined as “optionally substituted” the definition is meant to encompass when the groups is substituted with one or more of the radicals defined below, and when it is not so substituted.

Substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —N(R⁸⁰)₂, perhaloalkyl, —CN, —OCN, —SCN, —NO, —NO₂, ═N₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —P(O)(O⁻)₂(M⁺)₂, —P(O)(O⁻)₂M²⁺, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, −CO₂R⁷⁰, —C(S)OR⁷⁰, —C(O)N(R⁸⁰)₂, —C(NR⁷⁰)(R⁸⁰)₂, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ and —NR⁷⁰C(NR⁷⁰)N(R⁸⁰)₂, where R⁶⁰ is C₁₋₆alkyl, 3 to 10-membered heterocyclyl, 3 to 10-memberedheterocyclylC₁₋₆alkyl, C₆₋₁₀aryl or C₆₋₁₀arylC₁₋₆alkyl; each R⁷⁰ is independently for each occurence hydrogen or R⁶⁰; each R⁸⁰ is independently for each occurence R⁷⁰ or alternatively, two R⁸⁰'s, taken together with the nitrogen atom to which they are bonded, form a 3 to 7-membered heteroalicyclyl which optionally includes from 1 to 4 of the same or different additional heteroatoms selected from O, N and S, of which N optionally has H or C₁-C₃alkyl substitution; and each M⁺ is a counter ion with a net single positive charge. Each M⁺ is independently for each occurence, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (a “subscript 0.5 means e.g. that one of the counter ions for such divalent alkali earth ions can be an ionized form of a compound described herein and the other a typical counter ion such as chloride, or two ionized compounds can serve as counter ions for such divalent alkali earth ions, or a doubly ionized compound can serve as the counter ion for such divalent alkali earth ions). As specific examples, —N(R⁸⁰)₂ is meant to include —NH₂, —NH-alkyl, —NH-pyrrolidin-3-yl, N-pyrrolidinyl, N-piperazinyl, 4N-methyl-piperazin-1-yl, N-morpholinyl and the like.

Substituent groups for replacing hydrogens on unsaturated carbon atoms in groups containing unsaturated carbons are, unless otherwise specified, —R⁶⁰, halo, —O⁻ M⁺, —OR⁷⁰, —SR⁷⁰, —S⁻M⁺, —N(R⁸⁰)₂, perhaloalkyl, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —PO₃⁻²M²⁺, —P(O)(OR⁷⁰)O⁻M⁺, —P(O)(OR⁷⁰)₂, —C(O)R⁷⁰, —C(S)R)⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)N(R⁸⁰)₂, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ and —NR⁷⁰C(NR⁷⁰N(R⁸⁰)₂, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

Substituent groups for replacing hydrogens on nitrogen atoms in groups containing such nitrogen atoms are, unless otherwise specified, —R⁶⁰, —O⁻M⁺, —OR⁷⁰, —SR⁷⁰, —S⁻M⁺, —N(R⁸⁰)₂, perhaloalkyl, —CN, —NO, —NO₂, —S(O)₂R⁷⁰, —SO₃⁻M⁺, —SO₃R⁷⁰, —OS(O)₂R⁷⁰, —OSO₃⁻M⁺, —OSO₃R⁷⁰, —PO₃²⁻(M⁺)₂, —PO₃²⁻M²⁺, —P(O)(OR⁷⁰)O⁻M⁺, —P(O)(OR⁷⁰)(OR⁷⁰), —C(O)R⁷⁰, —C(S)R⁷⁰, —C(NR⁷⁰R⁷⁰, —CO₂R⁷⁰, —C(S)OR⁷⁰, —C(O)NR⁸⁰R⁸⁰, —C(NR⁷⁰)NR⁸⁰R⁸⁰, OC(O)R⁷⁰, —OC(S)R⁷⁰, —OCO₂R⁷⁰, —OC(S)OR⁷⁰, —NR⁷⁰C(O)R⁷⁰, —NR⁷⁰C(S)R⁷⁰, —NR⁷⁰CO₂R⁷⁰, —NR⁷⁰C(S)OR⁷⁰, —NR⁷⁰C(O)N(R⁸⁰)₂, —NR⁷⁰C(NR⁷⁰)R⁷⁰ and —NR⁷⁰C(NR⁷⁰)N(R⁸⁰)₂, where R⁶⁰, R⁷⁰, R⁸⁰ and M⁺ are as previously defined.

In one embodiment, a group that is substituted has 1, 2, 3, or 4 substituents, 1, 2, or 3 substituents, 1 or 2 substituents, or 1 substituent.

It is understood that in all substituted groups, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such case that the language permits such multiple substitutions, the maximum number of such iterations of substitution is three.

“Sulfonamide” refers to the group —SO₂NH₂, —N(H)SO₂H, —N(H)SO₂alkyl, —N(H)SO₂aryl, or —N(H)SO₂heterocyclyl.

“Sulfonyl” refers to the group —SO₂H, —SO₂alkyl, —SO₂aryl, or —SO₂heterocyclyl.

“Sulfanyl” refers to the group: —SH, —S-alkyl, —S-aryl, or —S-heterocyclyl.

“Sulfinyl” refers to the group: —S(O)H, —S(O)alkyl, —S(O)aryl or —S(O)heterocyclyl.

“Suitable leaving group” is defined as the term would be understood by one of ordinary skill in the art; that is, a group on a carbon, where upon reaction a new bond is to be formed, the carbon loses the group upon formation of the new bond. A typical example employing a suitable leaving group is a nucleophilic substitution reaction, e.g., on a sp³ hybridized carbon (SN₂ or SN₁), e.g. where the leaving group is a halide, such as a bromide, the reactant might be benzyl bromide. Another typical example of such a reaction is a nucleophilic aromatic substitution reaction (SNAr). Another example is an insertion reaction (for example by a transition metal) into the bond between an aromatic reaction partner bearing a leaving group followed by reductive coupling. “Suitable leaving group” is not limited to such mechanistic restrictions. Examples of suitable leaving groups include halogens, optionally substituted aryl or alkyl sulfonates, phosphonates, azides and —S(O)₀₋₂R where R is, for example optionally substituted alkyl, optionally substituted aryl, or optionally substituted heteroaryl. Those of skill in the art of organic synthesis will readily identify suitable leaving groups to perform a desired reaction under different reaction.

“Stereoisomer” and “stereoisomers” refer to compounds that have the same atomic connectivity but different atomic arrangement in space. Stereoisomers include cis-trans isomers, E and Z isomers, enantiomers and diastereomers. Compounds described herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)- isomers can be prepared using chiral synthons, chiral reagents, or resolved using conventional techniques, such as by: formation of diastereoisomeric salts or complexes which can be separated, for example, by crystallization; via formation of diastereoisomeric derivatives which can be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer can be synthesized by asymmetric synthesis using optically active reagents, binding partners, catalysts or solvents, or by converting on enantiomer to the other by asymmetric transformation. For a mixture of enantiomers, enriched in a particular enantiomer, the major component enantiomer can be further enriched (with concomitant loss in yield) by recrystallization.

When the compounds described herein contain olefinic double bonds or other centers of geometric asymmetry, and unless specified otherwise, it is intended that the compounds include both E and Z geometric isomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible and contemplated herein.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.).

“Prodrug” refers to compounds that are transformed in vivo to yield the parent compound, for example, by hydrolysis in the gut or enzymatic conversion in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) where the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention can be prepared according to conventional methods. A thorough discussion of prodrugs is provided in T. Higuchi and V. Stella, “Pro-drugs as Novel Delivery Systems,” Vol 14 of the A. C. S. Symposium Series, and in Bioreversible Carriers in Drug Design, ed. Edward B. Roche, American Pharmaceutical Association and Pergamon Press, 1987, both of which are incorporated herein by reference for all purposes.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 8^{th} Ed., Pergamon Press, Gilman et al. (eds), 1990 which is herein incorporated by reference). The metabolite of a compound described herein or its salt can itself be a biologically active compound in the body. While a prodrug described herein would meet this criteria, that is, form a described biologically active parent compound in vivo, “metabolite” is meant to encompass those compounds not contemplated to have lost a progroup, but rather all other compounds that are formed in vivo upon administration of a compound described herein which retain the biological activities described herein. Thus one aspect of the invention is a metabolite of a compound described herein. For example, a biologically active metabolite is discovered serendipitously, that is, no prodrug design per se was undertaken. Stated another way, biologically active compounds inherently formed as a result of practicing methods of the invention, arecontemplated and disclosed herein. “Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. The compounds described herein can exist in unsolvated as well as solvated forms with solvents, pharmaceutically acceptable or not, such as water, ethanol, and the like. Solvated forms of the presently disclosed compounds are contemplated herein and are encompassed by the invention, at least in generic terms.

An “antigen binding molecule,” as used herein, is any molecule that can specifically or selectively bind to an antigen. A binding molecule may include an antibody or a fragment thereof. An anti-galectin-12 binding molecule is a molecule that binds to the galectin-12 antigen, such as an anti-galectin-12 antibody or fragment thereof. Other anti-galectin-12 binding molecules may also include multivalent molecules, multi-specific molecules (e.g., diabodies), fusion molecules, aptimers, avimers, or other naturally occurring or recombinantly created molecules. Illustrative antigen-binding molecules useful to the present methods include antibody-like molecules. An antibody-like molecule is a molecule that can exhibit functions by binding to a target molecule (See, e.g., Current Opinion in Biotechnology 2006, 17:653-658; Current Opinion in Biotechnology 2007, 18:1-10; Current Opinion in Structural Biology 1997, 7:463-469; Protein Science 2006, 15:14-27), and includes, for example, DARPins (WO 2002/020565), Affibody (WO 1995/001937), Avimer (WO 2004/044011; WO 2005/040229), and Adnectin (WO 2002/032925).

An “antibody” refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains, respectively. As used in this application, an “antibody” encompasses all variations of antibody and fragments thereof that possess a particular binding specifically, e.g., for tumor associated antigens. Thus, within the scope of this concept are full length antibodies, chimeric antibodies, humanized antibodies, human antibodies, unibodies, single domain antibodies or nanobodies, single chain antibodies (ScFv), Fab, Fab′, and multimeric versions of these fragments (e.g., F(ab′)₂) with the same binding specificity.

Typically, an immunoglobulin has a heavy and light chain. Each heavy and light chain contains a constant region and a variable region, (the regions are also known as “domains”). Light and heavy chain variable regions contain a “framework” region interrupted by three hypervariable regions, also called “complementarity-determining regions” or “CDRs”. The extent of the framework region and CDRs have been defined. See, Kabat and Wu, SEQUENCES OF PROTEINS OF IMMUNOLOGICAL INTEREST, U.S. Government Printing Office, NIH Publication No. 91-3242 (1991); Kabat and Wu, J Immunol. (1991) 147(5):1709-19; and Wu and Kabat, Mol Immunol. (1992) 29(9):1141-6. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

References to “VH” refer to the variable region of an immunoglobulin heavy chain, including an Fv, scFv, dsFv or Fab. References to “VL” refer to the variable region of an immunoglobulin light chain, including of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

The term “linker peptide” includes reference to a peptide within an antibody binding fragment (e.g., Fv fragment) which serves to indirectly bond the variable domain of the heavy chain to the variable domain of the light chain.

The term “specific binding” is defined herein as the preferential binding of binding partners to another (e.g., a polypeptide and a ligand (analyte), two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g., a randomly generated molecule lacking the specifically recognized site(s); or a control sample where the target molecule or antigen is absent).

With respect to antibodies of the invention, the term “immunologically specific” “specifically binds” refers to antibodies and non-antibody antigen binding molecules that bind to one or more epitopes of a protein of interest (e.g., galectin-12), but which do not substantially recognize and bind other molecules in a sample containing a mixed population of antigenic biological molecules.

The term “selectively reactive” refers, with respect to an antigen, the preferential association of an antibody, in whole or part, with a cell or tissue bearing that antigen and not to cells or tissues lacking that antigen. It is, of course, recognized that a certain degree of non-specific interaction may occur between a molecule and a non-target cell or tissue. Nevertheless, selective reactivity, may be distinguished as mediated through specific recognition of the antigen. Although selectively reactive antibodies bind antigen, they may do so with low affinity. On the other hand, specific binding results in a much stronger association between the antibody and cells bearing the antigen than between the bound antibody and cells lacking the antigen. Specific binding typically results in greater than 2-fold, preferably greater than 5-fold, more preferably greater than 10- or 20-fold and most preferably greater than 100-fold increase in amount of bound antibody (per unit time) to a cell or tissue bearing galectin-12 as compared to a cell or tissue lacking galectin-12.

The term “immunologically reactive conditions” includes reference to conditions which allow an antibody generated to a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. See, e.g., Harlow & Lane, Using Antibodies, A Laboratory Manual (1998), for a description of immunoassay formats and conditions. Preferably, the immunologically reactive conditions employed in the methods of the present invention are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The term “contacting” includes reference to placement in direct physical association.

The terms “conjugating,” “joining,” “bonding” or “linking” refer to making two polypeptides into one contiguous polypeptide molecule. In the context of the present invention, the terms include reference to joining an antibody moiety to an effector molecule (EM). The linkage can be either by chemical or recombinant means. Chemical means refers to a reaction between the antibody moiety and the effector molecule such that there is a covalent bond formed between the two molecules to form one molecule. Biodegradable linkers are also contemplated. See, e.g., Meng, et al., Biomaterials. (2009) 30(12):2180-98; Duncan, Biochem Soc Trans. (2007) 35(Pt 1):56-60; Kim, et al., Biomaterials. (2011) 32(22):5158-66; and Chen, et al., Bioconjug Chem. (2011) 22(4):617-24.

The term “in vivo” includes reference to inside the body of the organism from which the cell was obtained. “Ex vivo” and “in vitro” means outside the body of the organism from which the cell was obtained.

The phrase “malignant cell” or “malignancy” refers to tumors or tumor cells that are invasive and/or able to undergo metastasis, i.e., a cancerous cell.

It is understood that the above definitions are not intended to include impermissible substitution patterns (e.g., methyl substituted with 5 fluoro groups). Such impermissible substitution patterns are easily recognized by a person having ordinary skill in the art.

### BRIEF DESCRIPTION OF THE DRAWINGS

FIGS. 1A-C illustrate generation of Lgals12^{-/-} mice. (A) Homologous recombination of the targeting construct (top) with the wildtype Lgals12 allele (middle) results in the replacement of ˜6 kb of Lgals12 DNA, encompassing exon III through IX, with a neomycin resistance cassette (pGK-neo) (bottom). Corresponding homologous sequences in the targeting construct and the Lgals12 gene are depicted in blue (short arm) and red (long arm), respectively. (B) Proper targeting is identified by Southern blotting of transfected ES DNA after digestion with indicated restriction enzymes and hybridized to the 5′ external P0 probe. Results from a negative (2G1) and a positive (7H9) ES clones were shown. (C) Mouse tail DNA was genotyped by PCR with primers P3 and P4 for wildtype Lgals12 allele, and P3 and P2 for the mutant allele, respectively (top). Total protein was extracted from Lgals12^{+/+}, Lgals12^{+/-}, and Lgals12^{-/-} epididymal adipose tissue and analyzed by Western blotting for galectin-12 expression (bottom).

FIGS. 2A-D illustrate galectin-12 deficiency reduces adiposity in mice. (A) Comparison of weights of visceral (epididymal) and subcutaneous (inguinal) white adipose tissue in Lgals12^{+/+} and Lgals12^{-/-} mice (+/+, n=13; −/−, n=18), as well as interscapular brown adipose tissue (BAT) and body weight. (B) Linear regression analyses show that body weight and fat depot weight are highly correlated in Lgals12^{+/+} mice (epididymal, R2=0.586, p=0.002; inguinal, R2=0.887, p<0.0001), but not in Lgals12^{-/-} mice (epididymal, R2<0.00001, p=0.992; inguinal, R2=0.015, p=0.676). (C) Triglyceride contents and adipocyte numbers of epidydymal fat depots from Lgals12^{+/+} (n=5) and Lgals12^{-/-} (n=4) mice. (D) H&E staining of paraffin sections of epididymal fat depots from Lgals12^{+/+} and Lgals12^{-/-} mice (representative of four experiments). Average diameters of >200 isolated adipocytes were determined from their digital images with ImageJ software using 100-μm Polybead polystyrene microspheres (Polysciences) as references. Results are from 22-24 weeks old males. Asterisks denote statistical significance (p<0.05).

FIG. 3 illustrates expression of adipose genes in Lgals12^{-/-} adipose tissue. RNA were extracted from Lgals12^{+/+} and Lgals12^{-/-} epididymal fat depots and analyzed for the expression of adipose genes by real-time PCR. mRNA levels of a gene in Lgals12^{-/-} cells were expressed as fold changes over those of Lgals12^{+/+} cells.

FIGS. 4A-D illustrate that galectin-12 is a lipid droplet protein. (A) 3T3-L 1 adipocytes were incubated for 2 h in serum-free DMEM medium. Protein levels of extracellular (in conditioned medium) and intracellular (cell-associated) galectin-12 and adiponectin were determined by Western blotting using specific antibodies. (B) Lipid droplets (LD) were purified from 3T3-L1 adipocytes by density gradient centrifugation, and the remaining cellular components were separated by differential centrifugation into 5 fractions containing plasma membrane (PM), high-density microsomes (HDM), low-density microsomes (LDM), cytosol, and mitochondria/nuclei (M/N). Levels of galectin-12, Glut-4, and perilipin A were determined in each fraction by Western blotting with respective specific antibodies. Each lane represents samples from an equal number of cells. (C) Immunostaining of 3T3 adipocytes showing galectin-12 (red) and perilipin A (green, bottom) on lipid droplets (green, top). (D) 3T3-L1 cells were incubated in the presence (MDI+) or absence (MDI−) of the adipogenic hormone cocktail to induce adipocyte differentiation. At indicated time points, cells were extracted for analysis of galectin-12 expression by Western blotting. (E) 3T3-L1 cells at various periods during differentiation were stained for galectin-12 (red), lipid droplets (green) and the nuclei (blue). Data are representative of three experiments with similar results.

FIGS. 5A-B illustrate association of galectin-12 with lipid droplets is not inhibited by lactose. (A) Mouse adipocytes from epididymal fat depot were lyzed and the lysates were incubated with fetuin-agarose, in the absence (−) or presence (+) of 25 mM lactose. Bound proteins were eluted with SDS-sample buffer and galectin-12 was detected by Western blotting. (B) Lipd droplets were purified from mouse adipocyte homogenates in the absence (−) or presence (+) of 25 mM lactose. Galectin-12 was detected as in (A).

FIGS. 6A-C illustrate that galectin-12 deficiency results in elevated lipolysis in adipocytes. (A) and (B) Adipocytes were isolated from epididymal fat depots of Lgals12^{+/+} and Lgals12^{-/-} mice (n=3-6) on a regular diet ad libitum. Equal numbers of cells were incubated in the absence (A) or presence (B) of 0.1 μM of isoproterenol. Glycerol and non-esterified fatty acid (NEFA) released into the medium were measured at different time points of incubation at 37° C. (C) siRNA-mediated knockdown was performed by electroporating 3T3-L1 adipocytes with either control siRNA (Ctrl) or a combination of two galectin-12-specific siRNAs (sil2). Three days later, galectin-12 levels were analyzed by Western blotting, and lipolysis was determined by monitoring the release of fatty acids and glycerol 1.5 h after isoproterenol stimulation. Asterisks denote statistical significance (p<0.05). Similar results were observed in four (A and B) or three (C) experiments.

FIGS. 7A-B illustrate that lipolysis is elevated in Lgals12^{-/-} adipocytes. (A) and (B) Adipocytes were isolated from epididymal fat depots of Lgals12^{+/+} and Lgals12^{-/-} mice (n=3-6) on a regular diet ad libitum. Equal volume of packed cells were incubated in the absence (A) or presence (B) of 0.1 μM of isoproterenol. Glycerol and FFA released into the medium were measured at different time points of incubation at 37° C.

FIGS. 8A-D illustrate tissue lipid content, total ambulatory activity, energy expenditure, and food intake in Lgals12^{+/+} and Lgals12^{-/-} mice. (A) Lipid contents of liver and muscle were determined for Lgals12^{+/+} and Lgals12^{-/-} male mice (n=5 for each genotype), by the ethanolic-KOH saponification method. (B) Food intake of Lgals12^{+/+} (n=5) and Lgals12^{-/-} (n=6). (C and D) Total ambulatory activity and oxygen consumption rates (VO₂) were determined by indirect calorimetry during the dark (7:00 pm-7:00 am) and light (7:00 am-7:00 pm) periods in Lgals12^{+/+} and Lgals12^{-/-} male mice (n=5 for each genotype) on standard diet. (E and F) Basal (E) or isoproterenol-stimulated (F) oxygen consumption of isolated epididymal white adipocytes from Lgals12^{+/+} and Lgals12^{-/-} mice (n=5-7) measured with the BD Oxygen Biosensor System. Oxygen consumption in panel E is expressed as normalized relative fluorescence unit (NRFU). Results in panel F are the ratios of oxygen consumption by Lgals12^{+/+} adipocytes to that of Lgals12^{-/-} adipocytes. All animals were studied at 22-26 weeks of age. Asterisks denote statistical significance (p<0.05).

FIGS. 9A-G illustrate galectin-12 ablation promotes PKA phosphorylation of hormone-sensitive lipase (HSL) and association of phosphorylated HSL and adipocyte triglycerol lipase (ATGL) with lipid droplets as a result of elevated cAMP levels. (A) Adipocytes from the epididymal fat depots of Lgals12^{+/+} and Lgals12^{-/-} mice were incubated with indicated concentrations of isoproterenol before being separated into cytosol and fat cake. Lipolytic proteins in each fraction were analyzed by Western blotting with indicated antibodies. (B) Quantification of lipid droplet (LD)-associated p-HSL and HSL by densitometry of Western blots (n=3 for each genotype). (C) Immunofluorescence of adipocytes differentiated from MEFs shows elevated levels of phospho-HSL associated with lipid droplets in Lgals12^{-/-} adipocytes 15 min after treatment with 0.1 μM isoproterenol. (D) Quantification of LD-associated ATGL by densitometry of Western blots (n=3 for each genotype). (E) Adipocytes from Lgals12^{+/+} (n=3) and Lgals12^{-/-} (n=3) mice were incubated with indicated concentrations of isoproterenol for 5 min at 37° C. and intracellular cAMP levels were determined by ELISA. (F) Adipocytes from Lgals12^{+/+} and Lgals12^{-/-} mice (n=3-6) were treated with or without 0.1 mM 3-isobutyl-1-methylxanthine (IBMX) or 1 U/ml adenosine deaminase (ADA) for 1 h at 37° C. and lipolysis was determined by measuring glycerol release. (G) cAMP/dbcAMP-stimulated lipolysis in adipocytes from Lgals12^{+/+} and Lgals12^{-/-} mice (n=4). Asterisks denote statistical significance (p<0.05). Results are representative of three experiments.

FIGS. 10A-C illustrate (A) Adipocytes from Lgals12^{+/+} and −/− mice (n=3) were incubated with 1 U/ml ADA and increasing concentrations of PIA for 1 h at 37° C. and glycerol release was measured. Results were expressed as both absolute values and as % of glycerol release in the absence of PIA. (B) Adipocytes from Lgals12+/+ and Lgals12^{-/-} mice (n=3 each) were incubated with or without 10 μM cilostamide (Cilo) or 25 μM rolipram (Roli), individually or in combination, for 2 h at 37° C. Lipolysis was determined by measuring glycerol release. (C) Insulin suppression of lipolysis in adipocytes stimulated with 20 nM isoproterenol (% of maximal release in the absence of insulin). Asterisks denote statistical significance (p<0.05). Similar results were obtained in three experiments.

FIGS. 11A-E illustrate galectin-12 deficiency reduces insulin resistance and glucose intolerance associated with weight gain. (A) and (B) Area under the curve (AUC) was computed from the plot of blood glucose levels as a function of time after i.p. injection of Lgals12^{+/+} and Lgals12^{-/-} mice with insulin (A) or glucose (B). The AUC values, which reflect insulin resistance (A) or glucose intolerance (B), were then plotted as a function of body weight. Note that insulin resistance and glucose intolerance correlate with body weight in Lgals12^{+/+} mice (insulin resistance vs body weight, R2=0.583, p=0.002; glucose intolerance vs body weight, R2=0.353, p=0.032). Such correlation was absent in Lgals12^{-/-} mice (insulin resistance vs body weight, R2<0.001, p=0.933; glucose intolerance vs body weight, R2=0.004, p=0.829). (C) Changes in plasma insulin levels in mice weighing >30 g during the first hour of the glucose tolerance test. (D) and (E) Plots of insulin resistance and glucose intolerance, as described in panel A and B, respectively, as a function of adiposity. Asterisks denote statistical significance (p<0.05). Results are representative of three to four experiments.

FIGS. 12A-C illustrate the effects of galectin-12 ablation on diet-induced and genetic (ob/ob) obesity. (A) Growth curve of Lgals12^{+/+} and Lgals12^{-/-} male mice fed a high-fat diet starting 4 weeks of age. (B) Body weight and fat depot weight of Lgals12^{+/+} (n=7) and Lgals12^{-/-} (n=8) mice fed a high-fat diet for 22 weeks, before or after food deprivation for 24 hours. (C) Growth curve of Lgals12^{+/+}, Lgals12^{+/-}, and Lgals 12^{-/-} ob/ob female mice. (D) Reduced body weights and weights of periovarian and inguinal fat depots in 12-month old Lgals12^{-/-} ob/ob females (n=7) compared to their Lgals12^{+/+} counterparts (n=8). Lgals12^{+/-} mice (n=5) also exhibited reduced inguinal fat depot mass. Asterisks denote statistical significance (p<0.05 by unpaired two-tailed Student's t-test).

FIG. 13 illustrates a Kyte/Doolittle hydropathy plot of the amino acid sequence of galectin-12 in comparison with Kyte/Doolittle hydropathy plots for perilipin A and galectin-3. Plots were generated using a window size of 19 amino acids. Hydrophobic sequences are displayed below the zero line.

FIG. 14 illustrates preparation of an encoded focused library with a galactose moiety and two points of diversity (R1 and R2). The outer layer contains the library compound and the inner layer contains the two encoding blocks for R1 and R2. The synthetic schemes are as follows: i) 20% piperidine in DMF, rt, 30 min; ii) a mixture of 4-(chloromethyl)benzoic acid (2 equiv), N-Fmoc-3-piperidinecarboxylic acid (2 equiv), HOBt (4 equiv), and DIC (4 equiv); iii) 50% TFA in DCM, rt, 30 min; iv) N-Fmoc-3-amino-3-(2-fluoro-5-nitrophenyl) propionic acid (5 equiv), HOBt, and DIC in DMF, rt, 5 h; v) R1NH2 (3 equiv), DMAP (3 equiv), rt, overnight; yl) 20% piperidine in DMF, rt, 30 min; vii) R2NCS (3 equiv), DIC (3 equiv), overnight; viii) 2M SnCl2.2H2O in DMF, 3 h×2; ix) O-glycosylation of tyrosine, hydroxylproline, serine and threonine, and x=D- or unnatural amino acids (5 equiv), HOBt, and DIC in DMF, rt, 5 h; x) 20% piperidine in DMF, rt, 30 min. The grey circle represents the solid matrix (e.g., a bead) on which the library is built and which facilitates screening. The galactose moiety (II) can be substituted, e.g., with a lactose, oligo-lactose, polylactose or thiodigalactose.

FIGS. 15A-B illustrate synthetic routes for 12 OBOC small molecule libraries. For simplicity, φ[galactose] moiety is omitted from the scheme; the φ[galactose] moiety can be inserted as an R group or as a glycol-amino acid between the solid support and the heterocyclic scaffolding. The grey circle represents the solid matrix (e.g., a bead) on which the library is built and which facilitates screening.

FIG. 16 illustrates the structure of a small organic one-bead-one-compound (OBOC) library built without a sugar group, but containing a polycyclic scaffold on which the library of functional groups is connected. The grey circle represents the solid matrix (e.g., a bead) on which the library is built and which facilitates screening.

FIG. 17 illustrates core structures and functional groups-X for exemplary inhibitors of galectin-12.

### DETAILED DESCRIPTION

### 1. Introduction

The breakdown of triglycerides, or lipolysis, is a tightly controlled process that regulates fat mobilization in accord with an animal's energy needs. It is well established that lipolysis is stimulated by hormones that signal energy demand and is suppressed by the anti-lipolytic hormone insulin. Yet much still remains to be learned about regulation of lipolysis by intracellular signaling pathways in adipocytes. The present invention is based, in part, on the discovery that galectin-12, a member of a β-galactoside-binding lectin family preferentially expressed by adipocytes, functions as an intrinsic negative regulator of lipolysis. Galectin-12 is primarily localized on lipid droplets and regulates lipolytic protein kinase A (PKA) signaling by acting upstream of phosphodiesterase (PDE) activity to control cyclic adenosine monophosphate (cAMP) levels. Ablation of galectin-12 in mice results in increased adipocyte mitochondrial respiration, reduced adiposity, and ameliorated insulin resistance/glucose intolerance. The present invention is based, in part, on the discovery of the unique properties of this intracellular galectin that is localized to an organelle and performs an important function in lipid metabolism. These findings add to the significant functions exhibited by intracellular galectins, and have important therapeutic implications for human metabolic disorders.

### 2. Subjects Who May Benefit

Subjects who may benefit from a regime of inhibiting the activity of galectin-12 may have or be predisposed to having a disease that is associated with or caused by abnormal or aberrant expression of or overexpression of galectin-12, or is associated with normal galectin-12 expression. In some embodiments, the subject may be at genetic risk of developing a disease that is associated with or caused by abnormal or aberrant expression of or overexpression of galectin-12.

Illustrative disease conditions associated with or caused by abnormal or aberrant expression of or overexpression of galectin-12 include metabolic disorders (e.g., including obesity, type 2 diabetes, and metabolic disease), cardiovascular disease, renal disease, and mitochondrial diseases (e.g., including without limitation Luft disease, Leigh syndrome (Complex I, cytochrome oxidase (COX) deficiency, pyruvate dehydrogenase (PDH) deficiency), Alpers Disease, Medium-Chain Acyl-CoA Dehydrogenase Deficiency (MCAD), Short-Chain Acyl-CoA Dehydrogenase Deficiency (SCAD), Short-chain-3-hydroxyacyl-CoA dehydrogenase (SCHAD) deficiency, Very Long-Chain Acyl-CoA Dehydrogenase Deficiency (VLCAD), Long-chain 3-hydroxyacyl-CoA dehydrogenase (LCHAD) deficiency, glutaric aciduria II, lethal infantile cardiomyopathy, Friedreich ataxia, maturity onset diabetes of young, malignant hyperthermia, disorders of ketone utilization, mtDNA depletion syndrome, reversible cox deficiency of infancy, various defects of the Krebs cycle, pyruvate dehydrogenase deficiency, pyruvate carboxylase deficiency, fumarase deficiency, carnitine palmitoyl transferase deficiency, Mitochondrial Myopathy (muscle weakness) (MELAS), MERRF syndrome (or Myoclonic Epilepsy with Ragged Red Fibers), neuropathy, ataxia, and retinitis pigmentosa (NARP) syndrome, Myoneurogastrointestinal disorder and encephalopathy (MNGIE), Pearson Marrow syndrome, Kearns-Sayre-CPEO, Leber hereditary optic neuropathy (LHON), Aminoglycoside-associated deafness, Diabetes with deafness, Methylmalonic academia, Erythropoietic porphyria, Propionic academia, Acute intermittent porphyria, Variegate porphyria, Maple syrup urine disease, Nonketotic hyperglycinemia, Hereditary sideroblastic anemia, Ornithine Transcarbamylase (OTC) Deficiency and Carbamyl Phosphate Synthetase (CPS) Deficiency).

In various embodiments, the subject has or is at risk of developing a cancer associated with or caused by abnormal or aberrant expression of or overexpression of galectin-12. Illustrative cancers include without limitation acute myeloid leukemia type M3 (acute promyelocytic leukemia), melanoma, and neuroblastoma.

### 3. Inhibiting the Activity of Galectin-12

Galectin-12 activity can be inhibited at either or both the protein level or the transcriptional level. In various embodiments, an agent that inhibits the binding activity of a galectin-12 protein is administered. In some embodiments, an agent that inhibits the expression, e.g., the transcription and or translation of a galectin-12 protein is administered.

a. Galectin-12 Proteins to be Inhibited

Generally, the binding activity of a galectin-12 protein, e.g., having at least 80% sequence identity, e.g., at least about 85%, 90%, 91%, 92%, 93%, 94%, 95%, 96%, 97%, 98% or 99% sequence identity, to an amino acid sequence of GenBank Ref. Nos. to NP_{—}001136007.1 (isoform 1); NP_{—}149092.2 (isoform 2); NP_{—}001136008.1 (isoform 3); NP_{—}001136009.1 (isoform 4); or NP_{—}001136010.1 (isoform 5), is inhibited or reduced, thereby preventing, reducing, delaying or inhibiting one or more symptoms of a disease condition associated with or caused by abnormal or aberrant expression or overexpression of galectin-12, or inhibiting normal galectin-12 expression that is beneficial to improvement of disease. In various embodiments, the inhibitors of galectin-12 binding activity may target conserved domains of the protein, e.g., one or more of the conserved carbohydrate recognition domains, a sugar binding pocket, a dimerization interface region, a GLECT[cd00070] domain.

b. Inhibiting Galectin-12 Binding Activity

In one embodiment, the methods involve reducing, inhibiting or preventing one or more symptoms of a disease condition associated with or caused by abnormal or aberrant expression or overexpression of galectin-12 by inhibiting the binding activity of a galectin-12 protein. This also applies to diseases with normal galectin-12 expression. Preferably, the inhibitory agent specifically or preferentially inhibits the binding activity of a galectin-12 protein in comparison to inhibiting the activity of proteins other than galectin-12 (e.g., other galectin proteins, e.g. galectin-1, -2, -5, -7, -10, -13, -14, or -15).

Examples of agents capable of inhibiting binding activity include binding partner analogs, alkylating agents, and inhibitory nucleic acids (reviewed in Ferscht, Structure and Mechanism in Protein Science: A Guide to Enzyme Catalysis and Protein Folding, 3rd Edition, 1999, W.H. Freeman & Co.). The methods of decreasing, inhibiting or preventing galectin-12 activity can involve administering to a subject, including a mammal such as a human, a compound that is an analog of a binding partner for a galectin-12, including small organic compound or peptidomimetic binding partner analogs.

The preferred galectin-12 inhibitors have no other adverse effects on cellular metabolism, so that other cellular functions proceed while the specific reaction of galectin-12 activity is inhibited. The blocking agents are preferably relatively small molecules, thereby avoiding immunogenicity and allowing passage through the cell membrane. Ideally, they have a molecular weight of between about 100-2000 daltons, but may have molecular weights up to 5000 or more, depending upon the desired application. In most preferred embodiments, the inhibitors have molecular weights of between about 200-600 daltons.

The inhibitors of the present invention preferably have strong affinity for the target protein, so that at least about 60-70% inhibition of galectin-12 activity is achieved, more preferably about 75%-85% and most preferably 90%-95% or more. In some embodiments, the inhibitors will completely inhibit galectin-12 activity. The affinity of the galectin-12 for the inhibitor is preferably sufficiently strong that the dissociation constant, or Kᵢ, of the galectin-12-inhibitor complex is less than about 10⁻⁵ M, typically between about 10⁻⁶ and 10⁻⁸ M.

Inhibitors can be classified according a number of criteria. For example, they may be directly competitive or non-competitive. The inhibitor may bind to galectin-12 covalently or noncovalently. In competitive inhibition for kinetically simple systems involving a single binding partner, the galectin-12 can bind either the binding partner or the inhibitor, but not both. Typically, competitive inhibitors resemble the binding partner or the product(s) and bind the binding site of the galectin-12, thus blocking the binding partner from binding the binding site. Noncompetitive inhibitors allow galectin-12 to bind the binding partner at the same time it binds the inhibitor. Another possible category of inhibition is mixed or uncompetitive inhibition, in which the inhibitor affects the binding site.

1. Anti-Galectin-12 Antigen Binding Molecule Inhibitors

Antigen binding molecules that bind to and reduce or inhibit the binding activity of a galectin-12 can be non-antibody binding molecules, or antibodies and fragments thereof. The antigen binding molecules can bind to any region of galectin-12 that inhibits or reduces its binding activity, e.g., by interfering directly with its binding partner binding. In various embodiments, the antigen binding molecule binds conserved domains of the protein, e.g., one or more of the conserved carbohydrate recognition domains, a sugar binding pocket, a dimerization interface region, a GLECT[cd00070] domain.

a. Non-Antibody Antigen Binding Molecules

In various embodiments, the antigen binding molecule is a non-antibody binding protein. Protein molecules have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc. Natl. Acad. Sci. U.S.A. 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc. Natl. Acad. Sci. U.S.A. 96(5): 1898-1903 (1999)) discloses an antibody mimic based on a lipocalin scaffold (Anticalin®). Lipocalins are composed of a β-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies. Anticalins® are small, single chain peptides, typically between 160 and 180 residues, which provide several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell. MoI. Biol. 49(2):209-216 (2003)) discusses a methodology for reducing antibodies into smaller peptidomimetics, they term “antibody like binding peptidomimetics” (ABiP) which can also be useful as an alternative to antibodies.

Silverman et al. (Nat. Biotechnol. (2005), 23: 1556-1561) discloses fusion proteins that are single-chain polypeptides comprising multiple domains termed “avimers.” Developed from human extracellular receptor domains by in vitro exon shuffling and phage display the avimers are a class of binding proteins somewhat similar to antibodies in their affinities and specificities for various target molecules. The resulting multidomain proteins can comprise multiple independent binding domains that can exhibit improved affinity (in some cases sub-nanomolar) and specificity compared with single-epitope binding proteins. Additional details concerning methods of construction and use of avimers are disclosed, for example, in U.S. Patent App. Pub. Nos. 20040175756, 20050048512, 20050053973, 20050089932 and 20050221384.

In addition to non-immunoglobulin protein frameworks, antibody properties have also been mimicked in compounds comprising RNA molecules and unnatural oligomers (e.g., protease inhibitors, benzodiazepines, purine derivatives and beta-turn mimics) all of which are suitable for use with the present invention.

b. Anti-Galectin-12 Antibodies

In various embodiments, the antigen-binding molecule is an antibody or antibody fragment that binds to galectin-12 and inhibits the binding activity of galectin-12. Such anti-galectin-12 antibodies are useful for preventing, delaying, inhibiting and treating the progression of disease condition associated with and/or cause by abnormal expression or overexpression of galectin-12. This also applies to disease condition associated with normal galectin-12 expression.

An antibody or antibody fragment suitable for treating, mitigating, delaying and/or preventing a disease condition associated with and/or cause by abnormal expression or overexpression of galectin-12, or associate with normal galectin-12 expression in a subject is specific for at least one portion of the galectin-12 polypeptide, e.g., the one or more of the conserved carbohydrate recognition domains, a sugar binding pocket, a dimerization interface region, a GLECT[cd00070] domain, or region involved in interacting with its binding partners. For example, one of skill in the art can use peptides derived from such conserved domains of a galectin-12 to generate appropriate antibodies suitable for use with the invention.

Anti-galectin-12 antibodies for use in the present methods include without limitation, polyclonal antibodies, monoclonal antibodies, chimeric antibodies, humanized antibodies, human antibodies, and fragments thereof.

The preparation of polyclonal antibodies is well-known to those skilled in the art. See, for example, Green et al., Production of Polyclonal Antisera, in IMMUNOCHEMICAL PROTOCOLS (Manson, ed), pages 1-5 (Humana Press 1992), Coligan et al, Production of Polyclonal Antisera in Rabbits, Rats. Mice and Hamsters, in CURRENT PROTOCOLS IN IMMUNOLOGY, section 2 4 1 (1992), which are hereby incorporated by reference.

The preparation of monoclonal antibodies likewise is conventional. See, for example, Kohler & Milstem, Nature 256 495 (1975). Coligan et al., sections 2.5.1-2.6.7, Harlow et al, ANTIBODIES A LABORATORY MANUAL, page 726 (Cold Spring Harbor Pub 1988, and Harlow, USING ANTIBODIES A LABORATORY MANUAL, Cold Spring Harbor Laboratory Press, 1998), which are hereby incorporated by reference Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al. sections 2.7.1-2.7.12 and sections 2.9.1-2.9.3, Barnes et al., Purification of Immunoglobulin G (IgG), in METHODS IN MOLECULAR BIOLOGY, VOL 10, pages 79-104 (Humana Press 1992).

Methods of in vitro and in vivo multiplication of monoclonal antibodies is well-known to those skilled in the art Multiplication in vitro can be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo can be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Anti-galectin-12 antibodies can be altered or produced for therapeutic applications. For example, antibodies of the present invention can also be derived from subhuman primate antibody. General techniques for raising therapeutically useful antibodies in baboons can be found, for example, in Goldenberg et al., International Patent Publication WO 91/11465 (1991) and Losman et al., Int. J. Cancer 46:310 (1990), which are hereby incorporated by reference.

Alternatively, therapeutically useful anti-galectin-12 antibodies can be derived from a “humanized” monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi, et al., Proc. Nat'l Acad. Sci. USA 86:3833 (1989), which is hereby incorporated in its entirety by reference. Techniques for producing humanized monoclonal antibodies are described, for example, by Jones et al., Nature 321:522 (1986); Riechmann et al., Nature 332:323 (1988); Verhoeyen et al., Science 239:1534 (1988); Carter et al. Proc. Nat'l Acad. Sci. USA 89:4285 (1992); Sandhu, Crit. Rev. Biotech. 12:437 (1992); and Singer et al., J. Immunol. 150:2844 (1993), which are hereby incorporated by reference.

Anti-galectin-12 antibodies for use in the present methods also can be derived from human antibody fragments isolated from a combinatorial immunoglobulin library. See, for example, Barbas, et al., METHODS: A COMPANION TO METHODS IN ENZYMOLOGY, VOL. 2, page 119 (1991); Winter et al., Ann. Rev. Immunol. 12:433 (1994), which are hereby incorporated herein by reference. Cloning and expression vectors that are useful for producing a human immunoglobulin phage library can be obtained, for example, from STRATAGENE Cloning Systems (now Agilent Technologies).

In addition, anti-galectin-12 antibodies for the mitigation, delay, treatment and/or prevention of a disease condition associated with or caused by abnormal expression or overexpression of a galectin-12 protein can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994); Lonberg et al. Nature 368:856 (1994); and Taylor et al., Int. Immunol. 6:579 (1994), which are hereby incorporated by reference.

In various embodiments, the antibodies are human IgG immunoglobulin. As appropriate or desired, the IgG can be of an isotype to promote antibody-dependent cell-mediated cytotoxicity (ADCC) and/or complement-dependent cellular cytotoxicity (CDCC), e.g., human IgG1 or human IgG3.

Antibody fragments for use in the present methods can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a fragment denoted F(ab′)₂— This fragment can be further cleaved using a thiol reducing agent, and optionally a blocking group for the sulfhydryl groups resulting from cleavage of disulfide linkages, to produce Fab′ monovalent fragments. Alternatively, an enzymatic cleavage using pepsin produces two monovalent Fab′ fragments and an Fc fragment directly. These methods are described, for example, by Goldenberg, U.S. Pat. No. 4,036,945 and U.S. Pat. No. 4,331,647, and references contained therein. These patents are hereby incorporated in their entireties by reference. See also, Nisonhoff, et al., Arch. Biochem. Biophys. 89:230 (1960); Porter, Biochem. J. 73:119 (1959): Edelman et al., METHODS IN ENZYMOLOGY, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques can also be used, so long as the fragments bind to the antigen that is recognized by the intact antibody.

For example, Fv fragments comprise an association of VH and VL chains. This association can be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See. e.g. Sandhu, Crit. Rev Biotechnol. 1992; 12(5-6):437-62. In some embodiments, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFv are described, for example, by Whitlow et al, METHODS: A COMPANION TO METHODS IN ENZYMOLOGY, VOL. 2, page 97 (1991); Bird et al, Science 242:423-426 (1988); Ladner, et al, U.S. Pat. No. 4,946,778; Pack, et al, BioTechnology 11:1271 77 (1993); and Sandhu, supra.

Another form of an antibody fragment suitable for use with the methods of the present invention is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al, METHODS: A COMPANION TO METHODS IN ENZYMOLOGY, VOL. 2, page 106 (1991), iv. Small Organic Compounds.

In some embodiments, the anti-galectin-12 antibody is a single-domain antibody (sdAb) or a nanobody. A single-domain antibody or a nanobody is a fully functional antibody that lacks light chains; they are heavy-chain antibodies containing a single variable domain (VHH) and two constant domains (CH2 and CH3). Like a whole antibody, single domain antibodies or nanobodies are able to bind selectively to a specific antigen. With a molecular weight of only 12-15 kDa, single-domain antibodies are much smaller than common antibodies (150-160 kDa) composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). Nanobodies are more potent and more stable than conventional four-chain antibodies which leads to (1) lower dosage forms, less frequent dosage leading to less side effects; and (2) improved stability leading to a broader choice of administration routes, comprising oral or subcutaneous routes and slow-release formulations in addition to the intravenous route. Slow-release formulation with stable anti-galectin-12 nanobodies, find use for the mitigation, delay, treatment and prevention of a disease condition associated with or caused by abnormal expression or overexpression of galectin-12, avoiding the need of repeated injections and the side effects associated with it. Because of their small size, nanobodies have the ability to cross membranes and penetrate into physiological compartments, tissues and organs not accessible to other, larger polypeptides and proteins.

Preferably, the antibodies are humanized for use in treating or preventing disease conditions in humans.

c. Anti-Galectin-12 Aptamers and Intramers

The inhibitor of galectin-12 expression or function may also comprise an aptamer. In the context of the present invention, the term “aptamer” comprises nucleic acids such as RNA, ssDNA (ss=single stranded), modified RNA, modified ssDNA or peptide nucleic acids (PNAs) which bind a plurality of target sequences having a high specificity and affinity. Aptamers are well known in the art and, inter alia, described in Famulok (1998) Curr. Op. Chem. Biol. 2:320-327. The preparation of aptamers is well known in the art and may involve, inter alia, the use of combinatorial RNA libraries to identify binding sites (Gold (1995) Ann. Rev. Biochem. 64:763-797).

Accordingly, aptamers are oligonucleotides derived from an in vitro evolution process called SELEX (systematic evolution of ligands by exponential enrichment). Pools of randomized RNA or single stranded DNA sequences are selected against certain targets. The sequences of tighter binding with the targets are isolated and amplified. The selection is repeated using the enriched pool derived from the first round selection. Several rounds of this process lead to winning sequences that are called “aptamers”. Aptamers have been evolved to bind proteins which are associated with a number of disease states. Using this method, many powerful antagonists of such proteins can be found. In order for these antagonists to work in animal models of disease and in humans, it is normally necessary to modify the aptamers. First of all, sugar modifications of nucleoside triphosphates are necessary to render the resulting aptamers resistant to nucleases found in serum. Changing the 2′OH groups of ribose to 2′F or 2′NH2 groups yields aptamers which are long lived in blood. The relatively low molecular weight of aptamers (8000-12000) leads to rapid clearance from the blood. Aptamers can be kept in the circulation from hours to days by conjugating them to higher molecular weight vehicles. When modified, conjugated aptamers are injected into animals, they inhibit physiological functions known to be associated with their target proteins. Aptamers may be applied systemically in animals and humans to treat organ specific diseases (Ostendorf (2001) J Am Soc Nephrol. 12:909-918). The first aptamer that has proceeded to phase I clinical studies is NX-1838, an injectable angiogenesis inhibitor that can be potentially used to treat macular degeneration-induced blindness. (Sun (2000) Curr Opin Mol Ther 2:100-105). Cytoplasmatic expression of aptamers (“intramers”) may be used to bind intracellular targets (Blind (1999) PNAS 96:3606-3610; Mayer (2001) PNAS 98:4961-4965). Said intramers are also envisaged to be employed in context of this invention.

ii. Inhibiting Galectin-12 Expression

Decreasing or inhibiting galectin-12 gene expression can be achieved using any method in the art, including through the use of inhibitory nucleic acids (e.g., small interfering RNA (siRNA), micro RNA (miRNA), antisense RNA, ribozymes, etc.). Inhibitory nucleic acids can be single-stranded nucleic acids that can specifically bind to a complementary nucleic acid sequence. By binding to the appropriate target sequence, an RNA-RNA, a DNA-DNA, or an RNA-DNA duplex or triplex is formed. Such inhibitory nucleic acids can be in either the “sense” or “antisense” orientation. See, for example, Tafech, et al., Curr Med Chem (2006) 13:863-81; Mahato, et al., Expert Opin Drug Deliv (2005) 2:3-28; Scanlon, Curr Pharm Biotechnol (2004) 5:415-20; and Scherer and Rossi, Nat Biotechnol (2003) 21:1457-65.

In one embodiment, the inhibitory nucleic acid can specifically bind to a target nucleic acid sequence or subsequence that encodes a human galectin-12. Administration of such inhibitory nucleic acids can decrease or inhibit the expression levels and consequently, the binding activity of galectin-12. Nucleotide sequences encoding a galectin-12 are known, e.g., NM_{—}001142535.1 (isoform 1); NM_{—}033101.3 (isoform 2); NM_{—}001142536.1 (isoform 3); NM_{—}001142537.1 (isoform 4); or NM_{—}001142538.1 (isoform 5). From these nucleotide sequences, one can derive a suitable inhibitory nucleic acid. Illustrative inhibitory nucleic acids for inhibiting the expression of galectin-12 are provided in U.S. Patent Publ. No. 2005/0250123 and herein.

1. Antisense Oligonucleotides

In some embodiments, the inhibitory nucleic acid is an antisense molecule. Antisense oligonucleotides are relatively short nucleic acids that are complementary (or antisense) to the coding strand (sense strand) of the mRNA encoding a galectin-12. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a galectin-12. Accordingly, antisense oligonucleotides decrease the expression and/or activity of galectin-12.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/09810) or the blood-brain barrier (see, e.g., PCT Publication No. WO 89/10134), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide can be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl)uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An anomeric oligonucleotide forms specific double-stranded hybrids with complementary RNA in which, contrary to the usual-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a galectin-12, one of skill in the art can design antisense oligonucleotides that bind to a target nucleic acid sequence and test these oligonucleotides in an in vitro or in vivo system to confirm that they bind to and mediate the degradation of the mRNA encoding the galectin-12. To design an antisense oligonucleotide that specifically binds to and mediates the degradation of a galectin-12 encoding nucleic acid, it is preferred that the sequence recognized by the oligonucleotide is unique or substantially unique to the galectin-12 to be inhibited. For example, sequences that are frequently repeated across an encoding sequence may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a galectin-12.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

2. Small Interfering RNA (siRNA or RNAi)

In some embodiments, the inhibitory nucleic acid is a small interfering RNA (siRNA or RNAi) molecule. RNAi constructs comprise double stranded RNA that can specifically block expression of a target gene. “RNA interference” or “RNAi” is a term initially applied to a phenomenon where double-stranded RNA (dsRNA) blocks gene expression in a specific and post-transcriptional manner. RNAi provides a useful method of inhibiting gene expression in vitro or in vivo. RNAi constructs can include small interfering RNAs (siRNAs), hairpin RNAs, and other RNA species which can be cleaved in vivo to form siRNAs. RNAi constructs herein also include expression vectors (“RNAi expression vectors”) capable of giving rise to transcripts which form dsRNAs or hairpin RNAs in cells, and/or transcripts which can produce siRNAs in vivo.

RNAi expression vectors express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell.

The RNAi constructs contain a nucleotide sequence that hybridizes under physiologic conditions of the cell to the nucleotide sequence of at least a portion of the mRNA transcript for the gene to be inhibited (i.e., a galectin-12-encoding nucleic acid sequence). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity can be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, for example, 95%, 96%, 97%, 98%, 99%, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodie-sters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO 01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO 01/68836 and WO 01/75164.

Exemplary RNAi constructs that specifically recognize a particular gene, or a particular family of genes can be selected using methodology outlined in detail above with respect to the selection of antisense oligonucleotide. Similarly, methods of delivery RNAi constructs include the methods for delivery antisense oligonucleotides outlined in detail above.

A program, siDESIGN from Dharmacon, Inc. (Lafayette, Colo.), permits predicting siRNAs for any nucleic acid sequence, and is available on the World Wide Web at dharmacon.com. Programs for designing siRNAs are also available from others, including Genscript (available on the Web at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

3. Ribozymes

In some embodiments, the inhibitory nucleic acid is a ribozyme. Ribozymes molecules designed to catalytically cleave an mRNA transcripts can also be used to prevent translation of mRNA (See, e.g., PCT International Publication WO 90/11364; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; WO 88/04300; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

b. Screening for Inhibitors of Galectin-12

One can identify lead compounds that are suitable for further testing to identify those that are therapeutically effective inhibitory agents by screening a variety of compounds and mixtures of compounds for their ability to decrease or inhibit galectin-12 binding activity in in vivo and in vitro assays, as described herein.

The use of screening assays to discover naturally occurring compounds with desired activities is well known and has been widely used for many years. For instance, many compounds with antibiotic activity were originally identified using this approach. Examples of such compounds include monolactams and aminoglycoside antibiotics. Compounds which inhibit various enzyme activities have also been found by this technique, for example, mevinolin, lovastatin, and mevacor, which are inhibitors of hydroxymethylglutamyl Coenzyme A reductase, an enzyme involved in cholesterol synthesis. Antibiotics that inhibit glycosyltransferase activities, such as tunicamycin and streptovirudin have also been identified in this manner.

Thus, another important aspect of the present invention is directed to methods for screening samples for inhibition or reduction of galectin-12 binding activity. A “sample” as used herein can be any mixture of compounds suitable for testing in a galectin-12 binding assay. A typical sample comprises a mixture of synthetically produced compounds or alternatively a naturally occurring mixture, such as a cell culture broth. Suitable cells include any cultured cells such as mammalian, insect, microbial or plant cells. Microbial cell cultures are composed of any microscopic organism such as bacteria, protozoa, yeast, fungi and the like.

In the typical screening assay, a sample, for example a fungal broth, is added to a standard galectin-12 binding assay. If inhibition or enhancement of activity as compared to control assays is found, the mixture is usually fractionated to identify components of the sample providing the inhibiting or enhancing activity. The sample is fractionated using standard methods such as ion exchange chromatography, affinity chromatography, electrophoresis, ultrafiltration, HPLC and the like. See, e.g., Scopes, Protein Purification, Principles and Practice, 3rd Edition, 1994, Springer-Verlag. Each isolated fraction is then tested for inhibiting or enhancing activity. If desired, the fractions are then further subfractionated and tested. This subfractionation and testing procedure can be repeated as many times as desired.

By combining various standard purification methods, a substantially pure compound suitable for in vivo therapeutic testing can be obtained. A substantially pure modulating agent as defined herein is an activity inhibiting or enhancing compound which migrates largely as a single band under standard electrophoretic conditions or largely as a single peak when monitored on a chromatographic column. More specifically, compositions of substantially pure modulating agents will comprise less than ten percent miscellaneous compounds.

In preferred embodiments, the assays are designed to screen large chemical libraries by automating the assay steps and providing compounds from any convenient source to assays, which are typically run in parallel (e.g., in microtiter formats on microtiter plates in robotic assays).

As noted, the invention provides in vitro assays for galectin-12 binding activity in a high throughput format. For each of the assay formats described, “no inhibitor” control reactions which do not include an inhibitory agent provide a background level of galectin-12 binding activity. In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many different plates per day; assay screens for up to about 6,000-20,000, and even up to about 100,000-1,000,000 different compounds is possible using the integrated systems of the invention. The steps of labeling, addition of reagents, fluid changes, and detection are compatible with full automation, for instance using programmable robotic systems or “integrated systems” commercially available, for example, through BioTX Automation, Conroe, Tex.; Qiagen, Valencia, Calif.; Beckman Coulter, Fullerton, Calif.; and Caliper Life Sciences, Hopkinton, Mass.

In some assays it will be desirable to have positive controls to ensure that the components of the assays are working properly. For example, a known inhibitor of galectin-12 binding activity can be incubated with one sample of the assay, and the resulting increase or decrease in signal determined according to the methods herein.

Essentially any chemical compound can be screened as a potential inhibitor of galectin-12 binding activity in the assays of the invention. Most preferred are generally compounds that can be dissolved in aqueous or organic (especially DMSO-based) solutions and compounds which fall within Lipinski's “Rule of 5” criteria. The assays are designed to screen large chemical libraries by automating the assay steps and providing compounds from any convenient source to assays, which are typically run in parallel (e.g., in microtiter formats on multiwell plates in robotic assays). It will be appreciated that there are many suppliers of chemical compounds, including Sigma-Aldrich (St. Louis, Mo.); Fluka Chemika-Biochemica Analytika (Buchs Switzerland), as well as numerous providers of small organic molecule libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), Tripos, Inc. (St. Louis, Mo.), Reaction Biology Corp. (Malvern, Pa.), Biomol Intl. (Plymouth Meeting, Pa.), TimTec (Newark, Del.), and AnalytiCon (Potsdam, Germany), among others.

In one preferred embodiment, inhibitors of galectin-12 binding activity are identified by screening a combinatorial library containing a large number of potential therapeutic compounds (potential inhibitor compounds). Such combinatorial chemical, nucleic acid or peptide libraries can be screened in one or more assays, as described herein, to identify those library members particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with .beta.-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

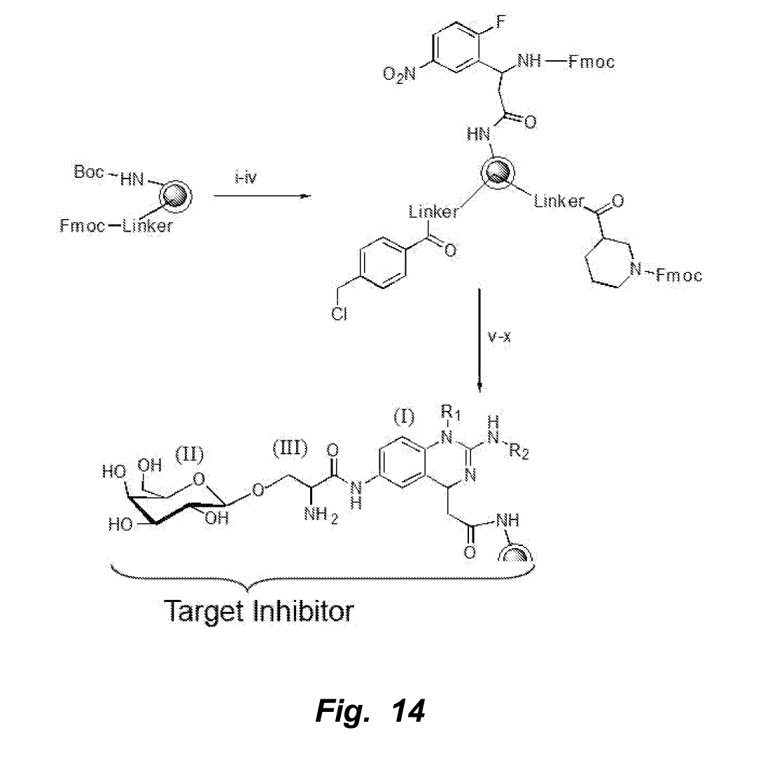

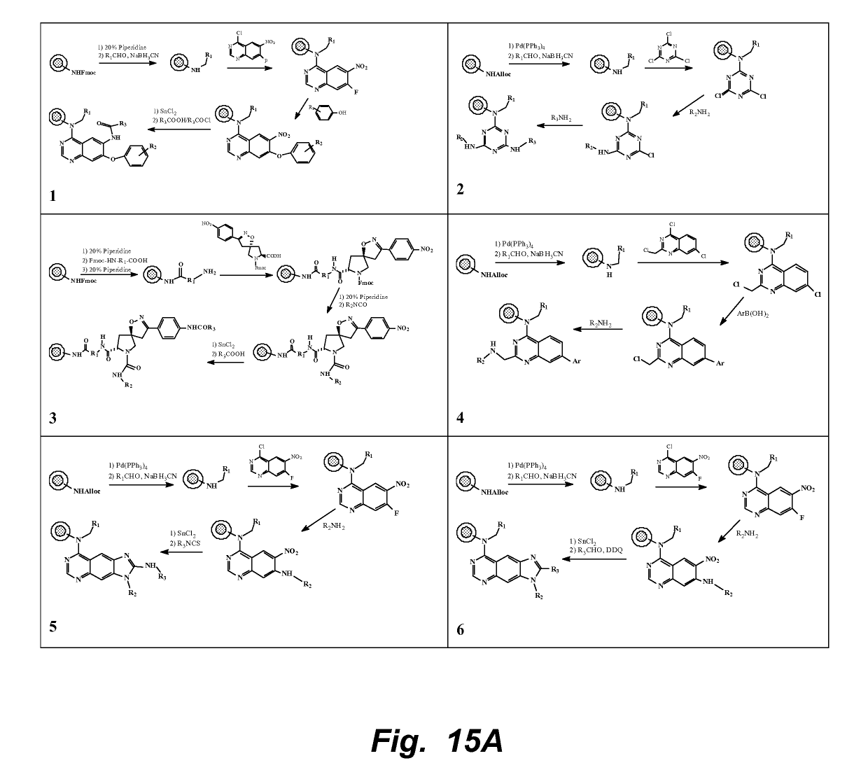

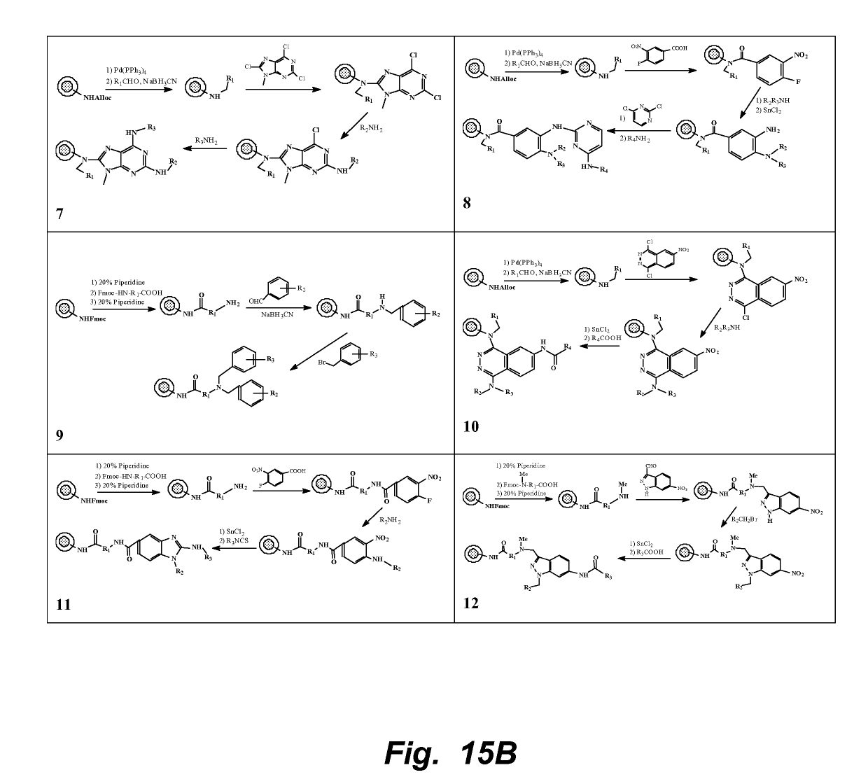

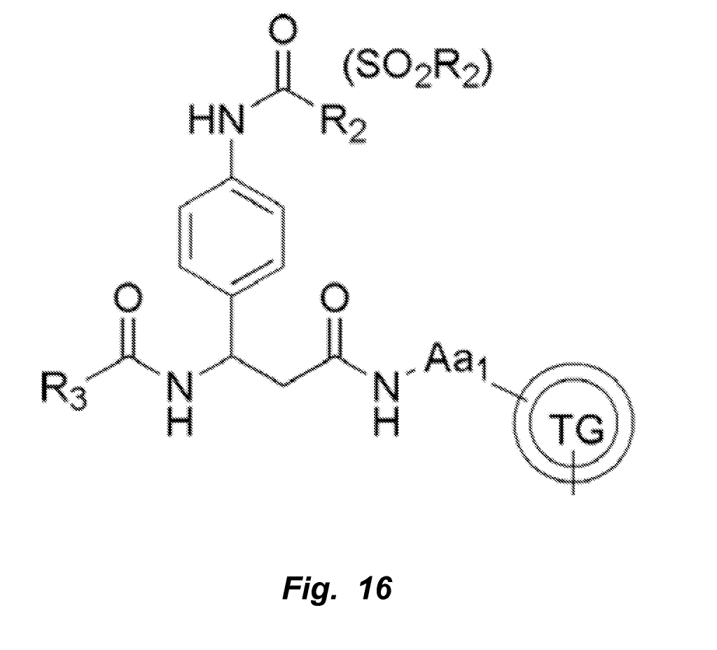

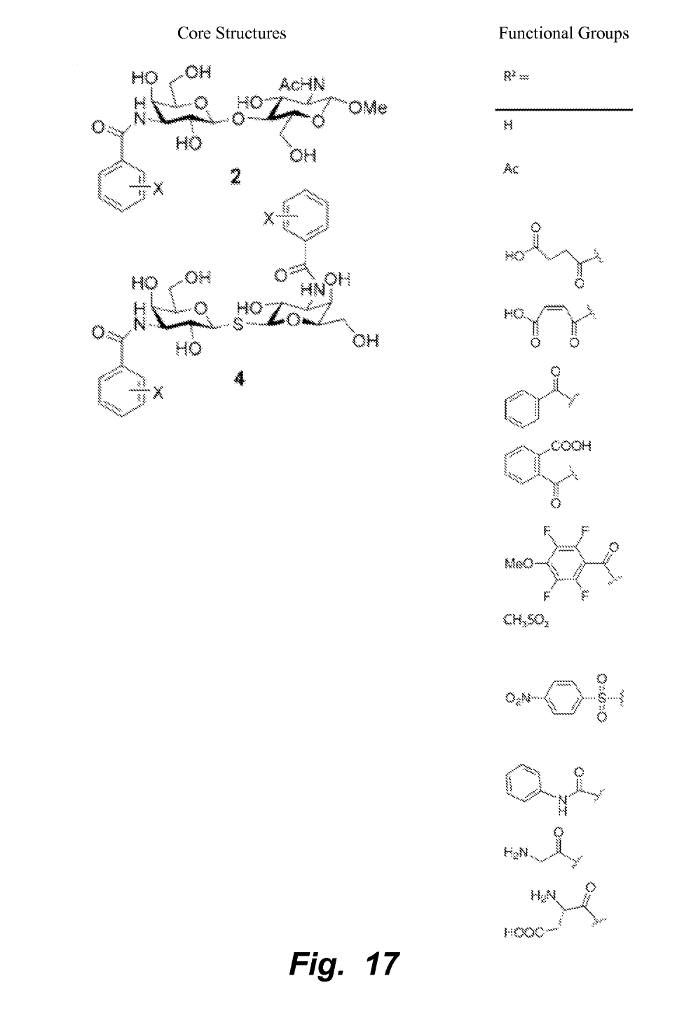

In various embodiments, the inhibitor of galectin-12 activity is identified in a library of compounds having a core structure as depicted in FIG. 14, FIG. 15A, FIG. 15B, FIG. 16, or FIG. 17. For example, in one embodiment, the inhibitor of galectin-12 activity is identified in a library of compounds having a substituted core structure selected from the following structures, wherein R1, R2, R3 and X are points of diversity and the grey circle represents the solid matrix (e.g., a bead) on which the library is built and which facilitates screening:

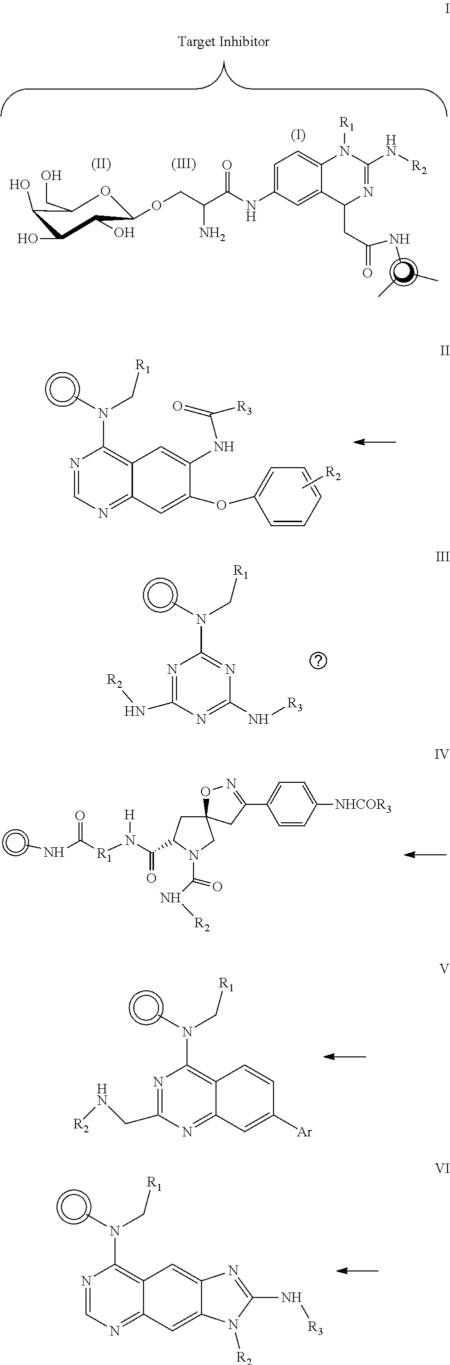

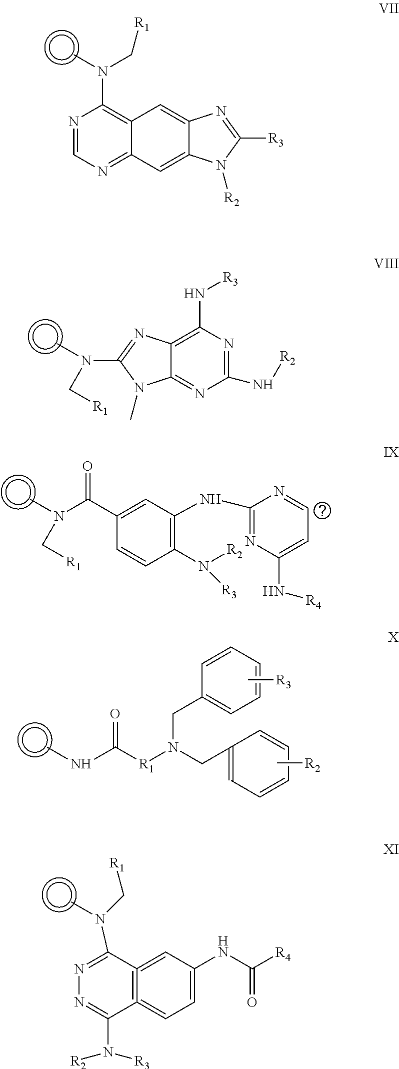

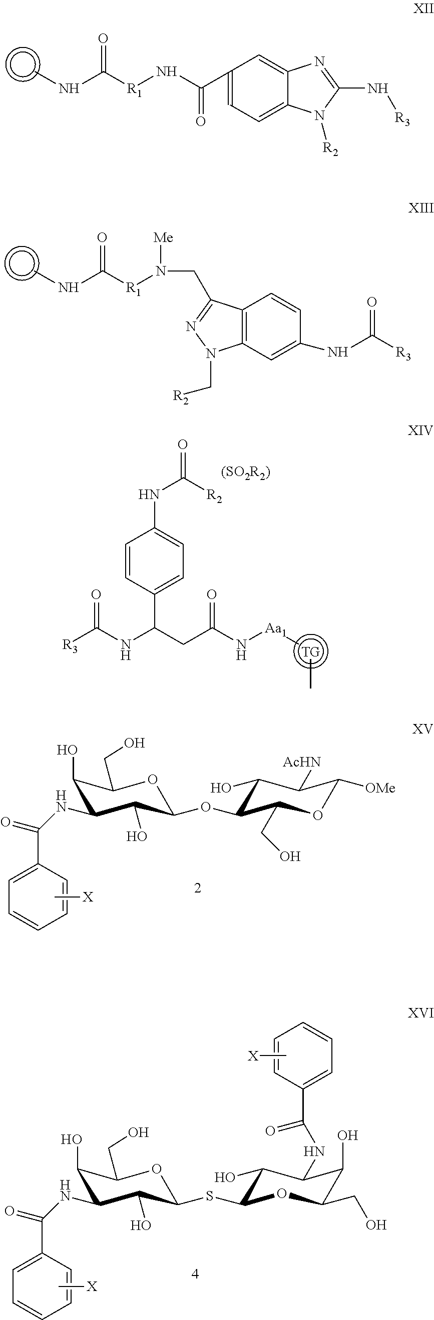

In some embodiments, the inhibitor of galectin-12 activity is identified in a library of compounds having a substituted core comprised of a galactose, lactose, an oligo-lactose, a poly-lactose, thiodigalactose, or analogs and/or derivatives thereof. In some embodiments, the inhibitor of galectin-12 activity is identified in a library of compounds having a galactose, a lactose, an oligo-lactose, a poly-lactose or a thiodigalactose nucleus attached to a scaffold comprising one or more linear, cyclic, aromatic, polycyclic linkers, wherein a library of functional groups is connected to the one or more linkers. In some embodiments, the inhibitor of galectin-12 activity is identified in a library of compounds having a N-Acetyl-lactosamine or a 3′-benzamido-N-Acetyl-lactosamine core. In some embodiments, the inhibitor of galectin-12 activity is identified in a library of compounds having a thiodigalactose or a 3,3′-bis-benzamido-thiodigalactoside core. Further galactin-12 inhibitors and libraries of galactin-12 inhibitors can be based on known galactin inhibitors, e.g., described in André, et al., Chembiochem (2001) 2:822-830; Glinsky, et al., Neoplasia. (2009) 11(9): 901-909; Iurisci, et al., Anticancer Research, (2009) 29(1):403-410; Cumpstey, et al., Angew Chem Int Ed Engl. (2005) 44(32):5110-2 and Sörme, et al., Chembiochem. (2002) 3(2-3):183-9.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.).

Lead compounds that have been identified for their capability to reduce or inhibit the binding activity of a galectin-12 in vitro are then evaluated for their ability to prevent, reduce or inhibit the ability of galectin-12 to bind to its binding partner in vivo and in vitro assays, as described herein, and/or to prevent or inhibit one or more symptoms associated with or caused by abnormal galectin-12 expression or galectin-12 overexpression. The ability of a particular compound to prevent, reduce or inhibit manifestations of disease in an animal model can be measured using any known technique. For example, test and control samples in in vitro assays and test and control animals in in vivo assays can be comparatively tested for disease signs. Assays for determining lipolysis, tissue lipid content, energy expenditure, food intake, ambulatory activity and oxygen consumption are described herein and known in the art.

### 4. Formulation and Administration

In therapeutic applications, the galectin-12 inhibitors can be administered to an individual already suffering from or at risk for developing a disease condition associated with or caused by abnormal expression or overexpression of galectin-12, or associated with normal galectin-12 expression that is beneficial to galectin-12 inhibition. Compositions that contain galectin-12 inhibitors are administered to a patient in an amount sufficient to suppress the undesirable metabolic and/or mitochondrial disorder and to eliminate or at least partially arrest symptoms and/or complications. An amount adequate to accomplish this is defined as a “therapeutically effective dose.” Amounts effective for this use will depend on, e.g., the inhibitor composition, the manner of administration, the stage and severity of the disease being treated, the weight and general state of health of the patient, and the judgment of the prescribing physician Inhibitors of galectin-12 activity can be administered chronically or acutely to treat or prevent a disease condition associated with or caused by abnormal expression or overexpression of galectin-12, or associated with normal galectin-12 expression that is beneficial to galectin-12 inhibition. In certain instances, it will be appropriate to administer an inhibitor of galectin-12 activity prophylactically, for instance in subjects at risk of or suspected of having a disease condition associated with or caused by abnormal expression or overexpression of galectin-12, or associated with normal galectin-12 expression that is beneficial to galectin-12 inhibition.

Alternatively, DNA or RNA that inhibits expression of one or more sequences encoding a galectin-12, such as a DNA or RNA aptamer, an antisense nucleic acid, a small-interfering nucleic acid (i.e., siRNA), a micro RNA (miRNA), or a nucleic acid that encodes a peptide that blocks expression or activity of a galectin-12 can be introduced into patients to achieve inhibition. U.S. Pat. No. 5,580,859 describes the use of injection of naked nucleic acids into cells to obtain expression of the genes which the nucleic acids encode.

Therapeutically effective amounts of galectin-12 inhibitor or enhancer compositions of the present invention generally range for the initial administration (that is for therapeutic or prophylactic administration) from about 0.1 μg to about 10 mg of galectin-12 inhibitor for a 70 kg patient, usually from about 1.0 μg to about 1 mg, for example, between about 10 μg to about 0.1 mg (100 μg). Typically, lower doses are initially administered and incrementally increased until a desired efficacious dose is reached. These doses can be followed by repeated administrations over weeks to months depending upon the patient's response and condition by evaluating symptoms associated with of a disease condition associated with or caused by abnormal expression or overexpression of galectin-12.

For prophylactic use, administration should be given to subjects at risk for or suspected of having a disease condition associated with or caused by abnormal expression or overexpression of galectin-12, or associated with normal galectin-12 expression that is beneficial to galectin-12 inhibition. Therapeutic administration may begin at the first sign of disease or the detection or shortly after diagnosis. This is often followed by repeated administration until at least symptoms are substantially abated and for a period thereafter.

The galectin-12 inhibitors for therapeutic or prophylactic treatment are intended for parenteral, topical, oral or local administration. Preferably, the compositions are formulated for oral administration. In certain embodiments, the pharmaceutical compositions are administered parenterally, e.g., intravenously, intranasally, inhalationally, subcutaneously, intradermally, or intramuscularly. Compositions of the invention are also suitable for oral administration. Thus, the invention provides compositions for parenteral administration which comprise a solution of the galectin-12 inhibiting agent dissolved or suspended in an acceptable carrier, preferably an aqueous carrier. A variety of aqueous carriers may be used, e.g., water, buffered water, 0.9% saline, 0.3% glycine or another suitable amino acid, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of galectin-12 inhibiting agents of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The galectin-12 inhibitors of the invention may also be administered via liposomes, which can be designed to target the conjugates to a particular tissue, for example lung, heart or CNS tissues, including brain tissue, as well as increase the half-life of the peptide composition. Liposomes include emulsions, foams, micelles, insoluble monolayers, liquid crystals, phospholipid dispersions, lamellar layers and the like. In these preparations, the peptide, nucleic acid or organic compound to be delivered is incorporated as part of a liposome, alone or in conjunction with a molecule which binds to, e.g., a receptor prevalent among the desired cells, or with other therapeutic compositions. Thus, liposomes filled with a desired peptide, nucleic acid, small molecule or conjugate of the invention can be directed to the site of, for example, immune cells, leukocytes, lymphocytes, myeloid cells or endothelial cells, where the liposomes then deliver the selected galectin-12 inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid liability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to desired cells, a ligand to be incorporated into the liposome can include, e.g., antibodies or fragments thereof specific for cell surface determinants of the target cells. A liposome suspension containing a galectin-12 inhibitor may be administered intravenously, locally, topically, etc. in a dose which varies according to, inter alia, the manner of administration, the conjugate being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more conjugates of the invention, and more preferably at a concentration of 25%-75%.

For aerosol administration, the inhibitors are preferably supplied in a suitable form along with a surfactant and propellant. Typical percentages of galectin-12 inhibitors are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

An effective treatment is indicated by a decrease in observed symptoms, as measured according to a clinician or reported by the patient. Alternatively, methods for detecting levels of specific galectin-12 activities can be used. Standard assays for detecting galectin-12 activity are described herein. Again, an effective treatment is indicated by a substantial reduction in activity of galectin-12. As used herein, a “substantial reduction” in galectin-12 activity refers to a reduction of at least about 30% in the test sample compared to an untreated control. Preferably, the reduction is at least about 50%, more preferably at least about 75%, and most preferably galectin-12 activity levels are reduced by at least about 90% in a sample from a treated mammal compared to an untreated control. In some embodiments, the galectin-12 activity is completely inhibited.

### EXAMPLES

The following examples are offered to illustrate, but not to limit the claimed invention.

### Example 1

### Ablation of a Galectin Preferentially Expressed in Adipocytes Increases Lipolysis, Reduces Adiposity, and Improves Insulin Sensitivity in Mice

### Materials and Methods

Generation of Lgals12^{-/-} mice.

We generated galectin-12-deficient mice in collaboration with the UC Davis Mouse Biology Program. The targeting construct was designed in such a way that after homologous recombination, the PGK-neo cassette would replace exon III-IX of the galectin-12 gene that constitutes most of the coding region.

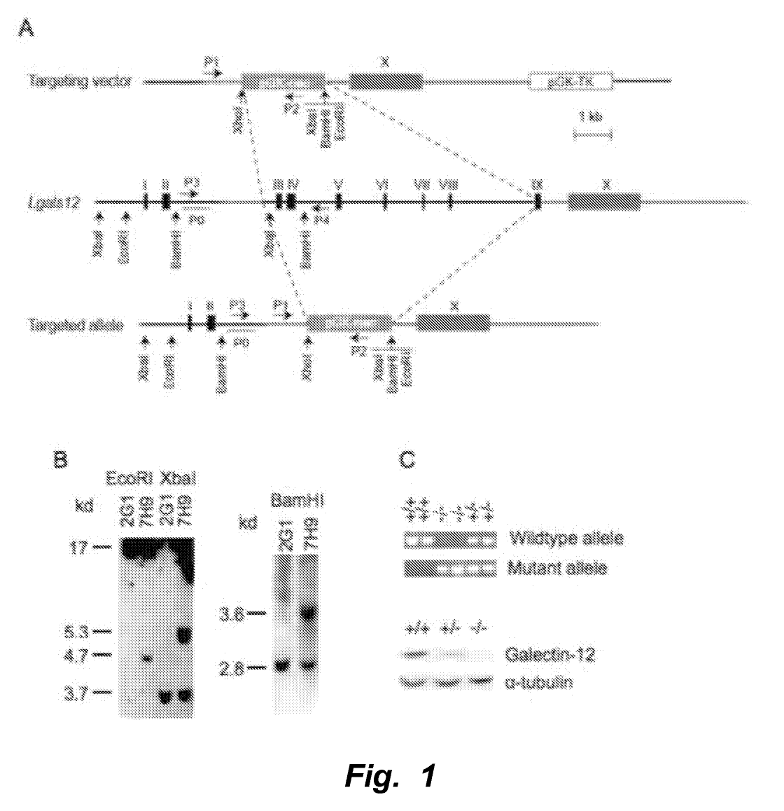

For construction of such targeting vector (FIG. 1), the long- and short arm fragments were amplified by PCR from mouse strain 129 genomic DNA with specific primers. They were then ligated to the backbone vector with the pGK-neo and pGK-TK cassettes following routine molecular cloning procedures. The resultant construct was verified by sequencing the ligation junctions, and transfected into mouse ES R1 cells by electroporation. Cells with stable insertion of the targeting cassette were selected by virtue of their survival in G418-containing medium. The selection medium also contains ganciclovir to eliminate cells with random insertions of the targeting vector, which are likely to retain the PGK-TK cassette and express the thymidine kinase gene. Thymidine kinase converts ganciclovir into a toxic product that kills the TK-expressing cells (Manis J P (2007) N Engl J Med 357:2426-2429).

Cells that survived the double selections were expanded to form colonies in 96-well plates. DNA was extracted from colonies in replicas of the plates with DNAzo1 (Invitrogen). We screened for the mutant allele with primers 5′-cagccagccagctcctgtacatgagggacc-3′ and 5′-gaacctgcgtgcaatccatcttgttcaatg-3′, using KlenTaq DNA polymerase (Ab Peptides) at the following conditions: One cycle at 94° C., 3 min; 5 cycles at 94° C. 30 s, 68° C. 30 s, 72° C. 3 min; 30 cycles at 94° C. 30 s, 60° C. 30 s, 72° C. 3 min.

Positive clones with a single PCR product of 1.6 kb were further confirmed by Southern blotting. The biotin-labeled probe for Southern blotting was synthesized by PCR using 0.05 mM biotin-16-dUTP (Roche Applied Science), 0.2 mM each of dATP, dCTG, dCTG, and 0.15 mM dTTP, with primers 5′-tagatggctagaagaatggaggtggattgc-3′ and 5′-ggtccctcatgtacaggagctggctggctg-3′. The PCR condition is 95° C. 2 min; 30 cycles at 95° C. 30 s, 60° C. 30 s, 72° C. 5 min. A control reaction with the omission of biotin-16-dUTP was also included. Products from both reactions were resolved by electrophoresis on agarose gel and the incorporation of biotin-16-dUTP is indicated by a significant shift of mobility. The biotin-labeled probe was purified using a PCR purification kit from Stratagene and quantified by UV spectrophotometry. DNA from PCR-positive clones was digested with appropriate restriction enzymes, separated by electrophoresis on 1% agarose gel and transferred to ZetaProbe GT Genomic Tested membranes (BioRad). Probe hybridization and chemiluminescence detection was carried out with the North2South® Chemiluminescent Hybridization and Detection Kit (Pierce), per the manufacturer's instructions.

ES clones with expected homologous recombination in the Lgals12 gene were injected into C57BL/6 blastocysts to obtain chimeric mice. Male animals with a high degree of coat color chimerism were bred to C57BL/6 females and germ line transmission was observed by coat pigment. Heterozygous mice were continuously backcrossed to C57BL/6J mice. The offspring was genotyped with regard to the galectin-12 gene by PCR of tail DNA with the above two primers for the mutant Lgals12 allele, as described for ES screening. After 8 generations of backcrossing, heterozygous males and females were intercrossed to generate littermates for experiments. They were genotyped by PCR with the primer pair as described for the Lgals12 mutant allele, and cagccagccagctcctgtacatgagggacc and cacactggaagtccacctgaaacctggtag for the wildtype allele (giving rise to a PCR product 1.7 kb), respectively.

Mouse Maintenance.

All animal studies were approved by the University of California at Davis Animal Care and Use Committee. Unless specified otherwise, mice used in these studies were from a C57BL/6J background after 8 generations of backcrossing and routinely fed a standard diet. Littermates of Lgals12^{+/+} and Lgals12^{-/-} male mice at ages 22-24 weeks were used in subsequent experiments, unless specified otherwise. For studies of high-fat induced obesity, starting 4 weeks of age, mice were fed a high-fat/high sugar (sucrose) diet in which 59.2% of energy is from fat, 25.7% from carbohydrates, and 15.2% from proteins (Formula 58R3, TestDiet).

Isolation of Primary Mouse Adipocytes.

Adipocytes were isolated from gonadal fat depots in Krebs-Ringer HEPES (KRH) buffer by collagenase digestion and floatation (Viswanadha S, Londos C (2006) J Lipid Res 47:1859-1864). After the final wash, cell density and triglyceride content of the adipocyte suspension were determined as described (Fine J B, DiGirolamo M (1997) Int J Obes Relat Metab Disord 21:764-768.).

Deconvolution Immunofluorescence Microscopy.

Mouse embryonic fibroblasts (MEFs) were isolated as described (Rosen E D et al. (2002) Genes Dev 16:22-26). Adipocyte differentiation of primary MEFs and 3T3-L1 fibroblasts was induced with an adipogenic hormone combination (Yang, et al., (2004) J Biol Chem 279:29761-29766). Cells were processed for immunostaining of cellular proteins as described (Ohsaki, et al., (2005) Histochem Cell Biol 124:445-452) Lipid droplets and nuclei were stained with 1 μg/mlBodipy 493/503 (Invitrogen) and 1 μg/ml Hoechst 33342 (Invitrogen), respectively. Fluorescent signals were visualized using an Olympus BX61 fluorescence microscope by capturing z-plane images at 1-μm intervals encompassing the depth of the cell. Flat-field-corrected image stacks were deconvolved by the constrained iterative method to mathematically remove out-of-focus light from the fluorescent image set using SlideBook 4.1 software (Intelligent Imaging Innovations).

Lipolysis Assay.

We monitored lipolysis in adipocytes isolated from random-fed Lgals12^{+/+} and Lgals12^{-/-} mice on regular diet, by measuring fatty acid and glycerol release, as described (Viswanadha S, Londos C (2006) J Lipid Res 47:1859-1864), with minor modifications. Briefly, 2.5×10⁵ cells were incubated in 0.3 ml KRH/3% FAA-free BSA (for basal lipolysis), or KRH/3% FAA-free BSA containing indicated concentrations of lipolysis stimulators or inhibitors (Sigma) for 0-2 h at 37° C. with shaking at 150 rpm. At the end of the incubation, glycerol and NEFA released into the infranatant was determined with the Free Glycerol Reagent (Sigma) and the Nonesterified Fatty Acids Kit (Catachem), respectively.

RNA Interference (RNAi) in 3T3-L1 Adipocytes.

3T3-L1 cells were induced to differentiate into adipocytes for 7 days as described (Yang, et al., (2004) J Biol Chem 279:29761-29766). We used the following doublestranded stealth siRNA oligonucleotides (Invitrogen) for RNA interference: set 1 for mouse galectin-12 (12#1), sense 5′-TTTACACTCACCTTCACCTCTTCGT-3′, antisense 5′-ACGAAGAGGTGAAGGTGAGTGTAAA-3′; set 2 for mouse galectin-12 (12#2), sense 5′-TAGCGGTAGTGAAGAAAGTGCTGGC-3′, antisense 5′-GCCAGCACTTTCTTCACTACCGCTA-3′. Control oligonucleotides with comparable GC content were also from Invitrogen. Cells were electroporated at 160 V, 1 mF with 2 nmol control oligonucleotides or a mixture of the two sets of galectin-12 siRNA oligonucleotides (1 nmol each) (Yang X et al. (2010) Cell Metab 11:194-205). Lipolysis was induced by treating cells for 1.5 h with 1 mM isoproterenol 3 days after transfection and assayed as described above.

Oxygen Consumption of Adipocytes.

Adipocytes were isolated from epididymal fat depots of Lgals12^{+/+} and Lgals12^{-/-} mice (22-24 weeks old) as described above and oxygen consumption by these cells was determined with BD Oxygen Biosensor System plates (BD Biosciences) (Wilson-Fritch L et al. (2004) J Clin Invest 114:1281-1289). An aliquot of the adipocytes were also subjected to the determination of DNA content (Gronblad M et al. (1996) Spine 21:2531-2538). Normalized relative fluorescence units (NRFU) were obtained by normalizing the fluorescence signal in each well of the Biosensor plate sequentially to pre-blank reading of the well, the signal in air-saturated buffer control, and the DNA content of the adipocyte sample (to normalize for cell number). Assay for isoproterenol-induced protein phosphorylation and translocation in adipocytes. After incubation of adipocytes with or without isoproterenol for 15 min with gentle shaking at 37° C., as described above, the incubation medium was remove from under the floating cell layer. Cells were incubated for 5 min on ice in 0.25 ml of permeabilization buffer (25 mM Tris-Phosphate pH 7.8, 10% glycerol, 2 mM EGTA, and 2 mM DTT, supplemented with 0.15 mg/ml digitonin). After centrifugation for 15 min at 13000 g at 4° C., the infranatant was collected as cytosol and lipid droplets were harvested in the fat cake. The fat cake was reconstituted in the same volume of buffer as the cytosol (0.25 ml) before 50 μl of 5×SDS-sample buffer was added to each fraction and proteins detected by Western blotting.

Intraperitoneal Insulin Sensitivity and Glucose Tolerance Assays.

To assess insulin sensitivity, insulin (Humulin-R, Lilly Diabetes) was injected i.p. into non-fasted mice (0.75 mU/g body weight). At different time points after injection, a small incision was made at the tail tip, and blood glucose levels were measured with a Glucometer (Bayer) by touching the blood drop coming out from the wound with a glucose strip. Glucose tolerance assay of C57BL/6J mice was performed after a 6-h fast. Blood glucose levels were measured as described above at different time points after i.p. injection of glucose (1.5 mg/g body weight).

Generation of Galectin-12 Antibodies.

Mouse galectin-12 cDNA was cloned in-frame into the pET-28 prokaryotic expression vector (Novagen). Expression of galectin-12 in E. coli strain BL-21 (DE3) was induced as described (Studier F W (2005) Protein Expression and Purification 41:207-234). Inclusion bodies that contain galectin-12 were purified (Singh S M, Panda Ak. (2005) J Biosci Bioeng 99:303-310) and used to immunize Lgals12^{-/-} mice. IgG was purified from immunized mouse sera by affinity chromatography on an rProtein A-Sepharose column (Biochain). The specificity of these antibodies was clearly demonstrated by their recognition of a single protein band with a molecular weight expected for galectin-12 on Western blots of adipocyte lysates from Lgals12^{+/+} but not Lgals12^{-/-} mice (FIG. 6 and FIG. 1).

Protein Extraction and Western Blotting.

For Western blotting, proteins were extracted from animal tissues or cells in an extraction buffer supplemented with protease and protein phosphatase inhibitors. After centrifuge at 13000 g, 4° C. for 10 min, protein concentrations of the supernatants were determined using the Pierce BCA Protein AssayKit (Thermo Scientific). Protein samples were denatured by boiling in SDS sample buffer, separated by SDS-PAGE, transferred to Immobilon-P membrane, and probed with indicated antibodies, as described (Yang, et al., J Biol Chem 279:29761-29766; Yang, et al., (2001) J Biol Chem 276:20252-20260).

Assay for Protein Secretion.

3T3-L1 cells on 12-well plates were induced to undergo adipocyte differentiation for 8 days (Yang, et al., J Biol Chem 279:29761-29766). Cells were washed and cultured in 1 ml serumfree medium for 2 h. Conditioned medium was recovered and centrifuged to remove insoluble material before being concentrated by precipitation with trichloroacetic acid. After final centrifugation, the pellet was resuspended in 30 μl SDS sample buffer. Cells were lysed in 0.1 ml SDS sample buffer. Samples (10 μl) from the conditioned media and cells were analyzed by Western blotting with galectin-12 and adiponectin antibodies (Millipore) to compare the extracellular and intracellular protein levels.

Determination of Food Intake, Energy Expenditure, and Tissue Triglyceride Content.

To determine the daily food intake, mice were singly housed with fresh chow of exact weight. The remainders of the chow were weighed the next day and the differences were considered as daily food intake. This process was repeated for three consecutive days to obtain the average amounts of daily food intake (Chen D et al. (2004) Mol Cell Biol 24:320-329). We measured oxygen consumption (VO₂) by indirect calorimetry using the Integra ME System (AccuScan Instruments, Inc., OH). The system also contains an activity analyzer for monitoring vertical and horizontal movements via light beam interruption to measure total ambulatory movement. Mice were put into 29×19×13 cm Plexiglas chambers and a 0.5 L/min flow rate was set for each chamber. Mice were acclimated to the chambers for 4-6 hours and studied for a 12-hour measurement during the dark cycle, followed by a 12-hour measurement during the light cycle. Ad libitum food and water were provided in the cages during the measurements. Energy expenditure was calculated as oxygen consumption normalized to body weight.

Tissue and Body Composition Analysis.

Liver and muscle triglyceride contents were determined by measuring glycerol release after saponification in ethanolic-KOH (Lau P et al. (2008) J Biol Chem 283:18411-18421). For body composition analysis, carcasses were homogenized in a IKA Ultra Turrax T25 high efficiency homogenizer in 50 ml distilled water. Total homogenate volume was measured and dry matter determined by drying duplicate 1-ml samples of homogenate overnight at 90° C. to stable weight. Total body lipid was determined by saponification of an aliquot of homogenate and measuring glycerol release with Sigma Glycerol Reagent, as described above.

Phosphodiesterase Assay.

Primary adipocytes were homogenized in HES buffer (20 mM HEPES, 1 mM EDTA, 0.25 M sucrose). The homogenates (0.1 ml) were diluted into 0.2 ml of assay buffer (50 mM HEPES, 10 mM MgCl₂) supplemented with 5 μM of the adenylyl cyclase inhibitor 2′,5′-dideoxyadenosine 3′-triphosphate. After incubation at 30° C. for 5 min with indicated selective PDE inhibitors, cAMP (0.6 μM) was added and the reaction was allowed to proceed for another 20 min. The reaction was stopped by the addition of 0.5 mM IBMX and centrifuged at 13000 g, 4° C. for 15 min. The remaining cAMP levels in the supernatant were determined as described above, and protein concentrations were measured with the Coomassie Plus Assay Kit (Pierce).

Assays for Serum Factors.

Serum levels of triglycerides and glycerol were determined with the Trinder kit from Sigma. Serum fatty acid levels were measured with the NEFAHR 2 kit (Wako). Insulin and leptin levels were assayed by ELISA with antibody pairs for each protein. For insulin, mouse anti-insulin mAb clone D6C4 (Advanced ImmunoChemical) was used as the capture antibody, and biotin-labeled mouse antiinsulin mAb clone D3E7 (Abcam) was used as the detection antibody. For leptin, the capture and detection antibodies used were purchased from R&D Systems.

Real-Time PCR.

Total RNA was isolated from cells using Trizol and reverse-transcribed with SuperScript III reverse transcriptase (Invitrogen). Real-time PCR was performed using the DNA dye EvaGreen (Biotium) and KlenTaq1 DNA polymerase (Ab Peptides) on the Applied Biosystems 7900HT Fast Real-Time PCR System. Data were collected and analyzed with Sequence Detection System (SDS) v2.4 software. Primer sequences are listed in Table 1.

TABLE 1
Oligonucleotide sequences for real-time PCR

| Gene | Primer pair |
|---|---|
| Adipsin | 5′-CTACCCTTGCAATACGAGGACAAAGAAGTG-3′ |
| | 5′-TCAGGATGTCATGTTACCATTTGTGATG-3′ |
| FAS | 5′-TCAGTGGAGGCAGGAGCCAAACTGAGC-3′ |
| | 5′-CACAGTCCAGACACTTCTTCACACTGAC-3′ |
| Leptin | 5′-CTGGCAGTCTATCAACAGGTCC-3′ |
| | 5′-TGTGGAGTAGAGTGAGGCTTCC-3′ |
| Adiponectin | 5′-TATGACGGCAGCACTGGCAAGTTCTACTGC-3′ |
| | 5′-ATGGGTAGTTGCAGTCAGTTGGTATCATGG-3′ |
| LPL | 5′-ATGTTAGAGAAGTAGTTCCAGATATGCTGG-3′ |
| | 5′-GACTGAGACAGACAATCATGGATGGAGACG-3′ |
| Cyclophilin A | 5′-CTGCACTGCCAAGACTGAATGGCTGGATGG-3′ |
| | 5′-GGACGCTCTCCTGAGCTACAGAAGGAATGG-3′ |
| aP2 | 5′-TGCCTTTGCCACAAGGAAAGTGGCAGGC-3′ |
| | 5′-TCCGACTGACTATTGTAGTGTTGATGC-3′ |
| C/EBP+ | 5′-GCTCTGATTCTTGCCAAACTGAGACTCTTC-3′ |
| | 5′-AGGAAGCTAAGACCCACTACTACATACACC-3′ |

Cyclophilin A (CPA) mRNA levels were taken as references. The mRNA levels of the target gene normalized to those of cyclophilin A gene were calculated based on the threshold cycle (Ct) as 2^{ΔCt}, where ΔCt=Ct_{CPA}−C_{target}.

Statistical Methods.

Data are presented as means±standard error (s.e.). Measurements in Lgals12^{+/+} and Lgals12^{-/-} mice were compared by unpaired two-tailed Student's t-tests using Prism 5 (GraphPad Software, Inc.). Results were considered statistically significant at p<0.05.

### Results

Galectin-12 Ablation Reduces Adiposity as a Result of Decreased Adipocyte Triglyceride Content.

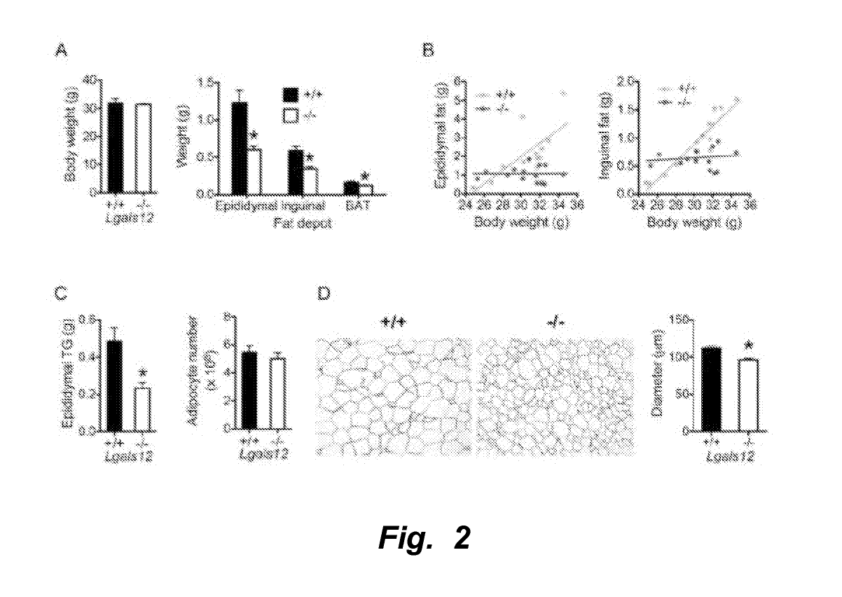

It has been previously reported that galectin-12 is preferentially expressed in adipose tissue and that its expression is regulated by hormones and cytokines that regulate insulin sensitivity (9,14), suggesting its involvement in energy homeostasis. To study the role of galectin-12 in energy metabolism in vivo, we generated galectin-12-deficient (Lgals12^{-/-}) mice (FIG. 51) and examined their adipose tissue phenotype. We found that Lgals12^{-/-} animals had substantially reduced visceral (epididymal) and subcutaneous (inguinal) white adipose tissue, despite normal body weights (FIG. 2A). Reduced adiposity of Lgals12^{-/-} mice was also supported by body composition analysis, which showed a ˜40% reduction in whole-body lipid content (Table 2).

TABLE 2
Body composition of Lgals12^{+/+} and Lgals12^{-/-} male mice at
21 weeks of age

| | Lgals12^{+/+} (n = 7) | Lgals12^{-/-} (n = 7) | p-value |
|---|---|---|---|
| Body weight (g) | 31.73 ± 0.481 | 29.58 ± 0.983 | 0.072 |
| Lipid (%) | 12.32 ± 1.842 | 7.552 ± 0.478 | 0.028* |
| Water (%) | 63.98 ± 1.770 | 68.97 ± 0.926 | 0.027* |
| Dry lean mass (%) | 23.71 ± 1.436 | 23.47 ± 0.976 | 0.896 |

Results are presented as mean ± s.e.m.
Asterisk indicates statistical significance.

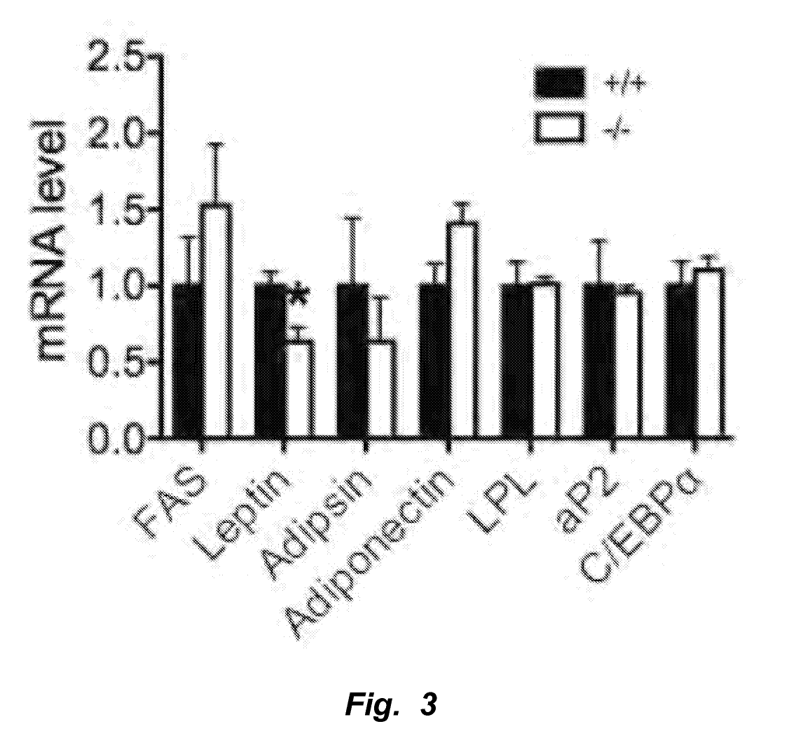

The strong positive correlation between body weights and weights of fat depots seen in Lgals12^{+/+} mice was not observed in Lgals12−/− mice (FIG. 2B). Epididymal fat depots of Lgals12^{-/-} mice contained less triglyceride compared to Lgals12^{+/+} mice, while the number of adipocytes was not significantly altered (FIG. 2C). Consistent with this, the adipocytes of Lgals12^{-/-} mice were smaller in size (FIG. 2D). These results indicate that the decrease in size of fat depots in Lgals12^{-/-} mice is due to a reduction of triglyceride content and not tissue cellularity. An early report suggested that galectin-12 is involved in adipogenesis in vitro (15). However, we did not observe major alterations in the expression of several adipose genes examined in Lgals12−/− mice, suggesting that adipose tissue development in these mice are largely normal (FIG. 3). Nevertheless, the levels of leptin expression in adipose tissue were significantly lower in these mice. This is likely due to reduced adiposity because leptin expression is regulated by adiposity (16).

Galectin-12 is Localized to Adipocyte Lipid Droplets.

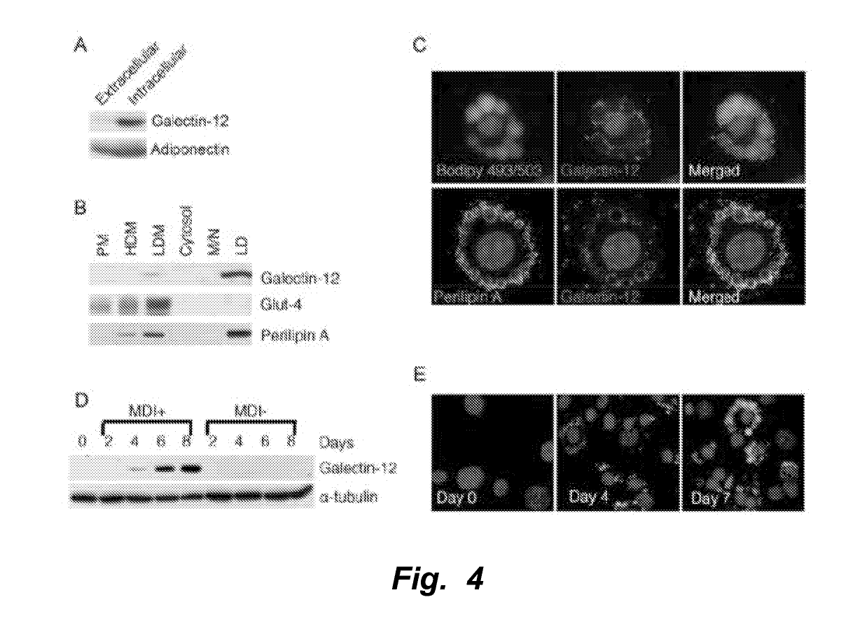

We generated mouse anti-galectin-12 antibodies and used them for cellular localization of galectin-12. The antibodies recognize a single protein band on Western blotting of protein extracts from Lgals12^{+/+} adipose tissue but not Lgals12^{-/-} adipose tissue (FIG. 1C, lower panel), establishing its specificity. Unlike adipokines, such as adiponectin, galectin-12 is not secreted in a significant amount under normal conditions (FIG. 4A). Using a well-established cellular fractionation method for 3T3-L1 adipocytes (17), we found that galectin-12 was detectable in the low-density microsomal (LDM) fraction, but the vast majority co-purified with lipid droplets (LD) (FIG. 4B), as did perilipin A, a known lipid droplet protein (18). This was further confirmed by co-staining with Bodipy 493/503, a fluorescent dye specific for neutral lipids in lipid droplets (FIG. 4C, top). Co-staining with anti-perilipin A antibody revealed that while perilipin A was found on both small and large lipid droplets, galectin-12 was mainly localized on large droplets (FIG. 4C, bottom).

Galectin-12 protein could be detected 4 days after induction of 3T3-L1 adipocyte differentiation. At this early stage, most lipid droplets were small and only a few larger droplets were coated with galectin-12. The levels of galectin-12 plateaued around one week into differentiation. At this mature stage, most lipid droplets were large and positive for galectin-12, but some remained small and negative for this protein (FIG. 4D, E).

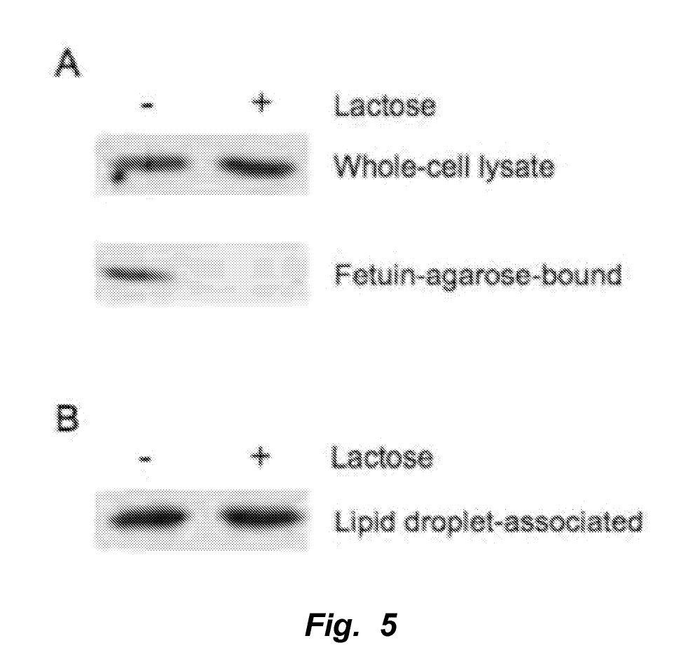

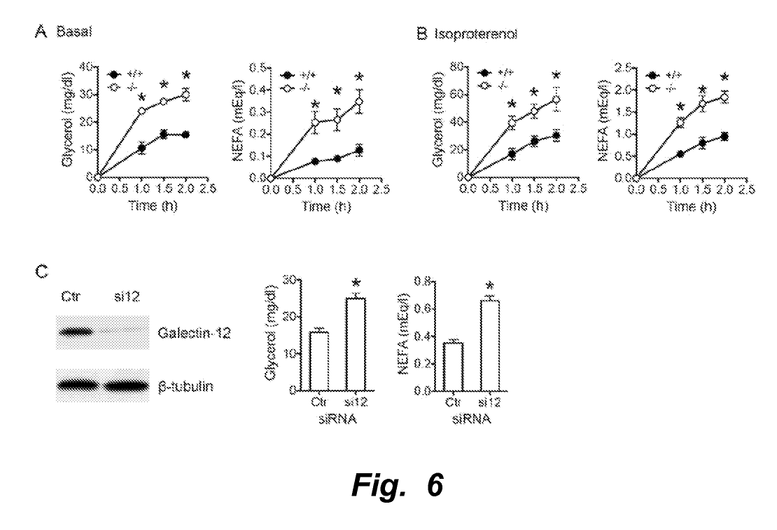

To test whether galectin-12 association with lipid droplets is glycan-dependent, we also purified lipid droplets in the presence of lactose. Lactose at a concentration of 25 mM, which completely inhibited the binding of galectin-12 to fetuin-agarose (FIG. 5A), did not affect galectin-12 association with lipid droplets (FIG. 5B), suggesting that the association is not likely to be mediated by glycans.

Galectin-12 Deficiency Enhances Lipolysis.

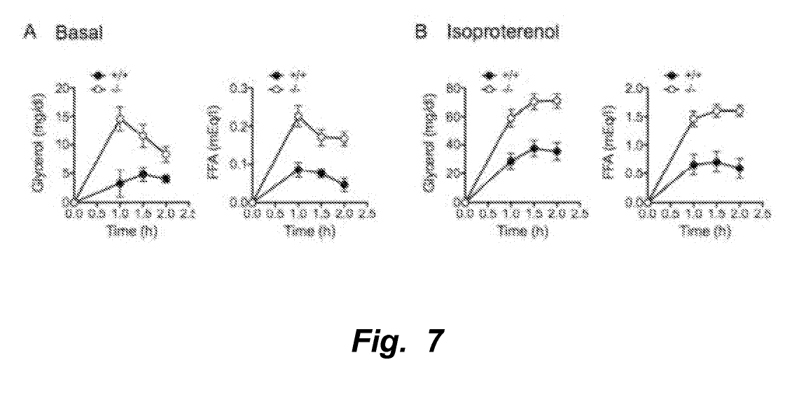

The lipid droplet protein perilipin A plays an important role in lipolysis. Similarly, we found that lipolysis in Lgals12^{-/-} adipocytes was approximately two fold higher compared to equal numbers of Lgals12^{+/+} cells, both under basal conditions (FIG. 6A) and when stimulated with the β-adrenergic receptor agonist isoproterenol (FIG. 6B). Even greater differences were observed when equal volumes of packed cells were compared (FIG. 7). We also used RNA interference to further confirm the effects of galectin-12 on lipolysis. Transfection of 3T3-L1 adipocytes with galectin-12 siRNAs significantly suppressed galectin-12 expression and enhanced isoproterenol-stimulated lipolysis (FIG. 6C). Negative control of lipolysis by galectin-12 is consistent with its specific localization to large lipid droplets in mature adipocytes (FIG. 4C), as centrally located, large lipid droplets are known to be less sensitive to lipolytic stimulation than those small, peripheral droplets (19).

Despite increased lipolysis, serum glycerol and fatty acid levels were not increased in Lgals12^{-/-} mice (Table 3).

TABLE 3

| | Lgals12^{+/+} (n = 7) | Lgals12^{-/-} (n = 6) | p-value |
|---|---|---|---|
| Triglyceride (mg/dl) | 156.1 ± 7.36 | 148.2 ± 11.75 | 0.565 |
| Glycerol (mg/dl) | 50.77 ± 6.934 | 38.25 ± 5.273 | 0.190 |
| NEFA (mEq/l) | 0.447 ± 0.105 | 0.363 ± 0.096 | 0.574 |
| Insulin (ng/ml) | 1.963 ± 0.556 | 1.448 ± 0.225 | 0.437 |
| Adiponectin | 10.86 ± 1.852 | 11.46 ± 0.969 | 0.781 |
| (mg/ml) | | | |
| Leptin (ng/ml) | 6.730 ± 1.491 | 2.751 ± 0.614 | 0.041* |

Results are presented as mean ± s.e.m.
Asterisk indicates statistical significance.

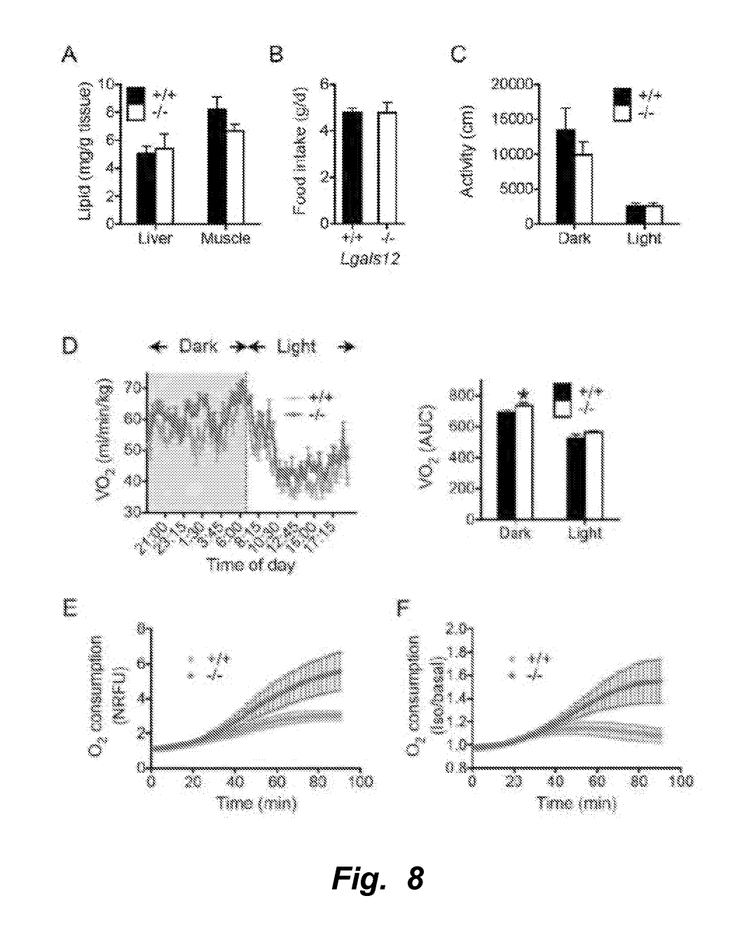

Lipid contents of liver and muscle were also comparable between Lgals12^{+/+} and Lgals12^{-/-} mice (FIG. 8A). There were no significant differences in food intake (FIG. 8B) or ambulatory activity (FIG. 8C) between the two genotypes, yet we observed increased oxygen consumption by Lgals12^{-/-} animals indicative of increased energy expenditure (FIG. 8D). In a similar fashion to white adipocytes deficient in the lipid droplet protein FSP27 (fat-specific protein of 27 kDa) (20,21) or the critical macroautophage gene Atg7 (autophagy-related 7) (22), Lgals12^{-/-} white adipocytes exhibited increased oxygen consumption (FIG. 4E). Induction of lipolysis with isoproterenol resulted in greater stimulation of oxygen consumption in Lgals12^{-/-} adipocytes compared to Lgals12^{+/+} cells (FIG. 8F). The results indicate that enhanced mitochondrial respiration in white adipocytes contributes to increased energy expenditure in Lgals12^{-/-} mice.

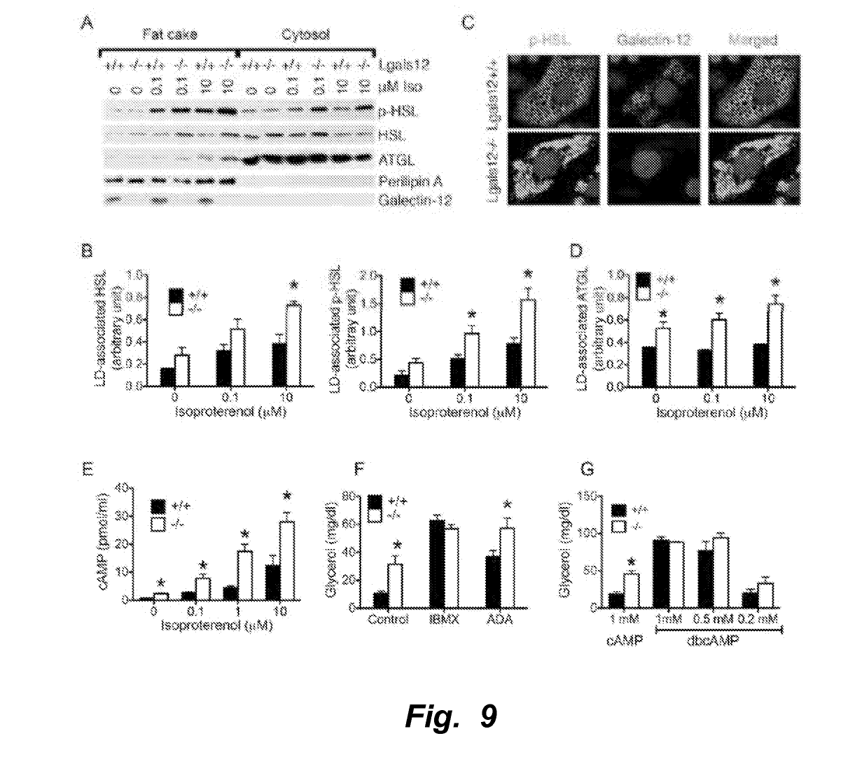

Lgals12^{-/-} adipocytes show elevated PKA phosphorylation of HSL and association of HSL and ATGL with lipid droplets. PKA phosphorylation is a critical event for the activation and recruitment of hormone-sensitive lipase (HSL) to lipid droplets (23,24), where it operates in concert with adipocyte triglyceride lipase (ATGL) to hydrolyze stored lipids (25-27). We treated primary adipocytes from Lgals12^{+/+} and Lgals12^{-/-} mice with isoproterenol, separated the cytosolic and fat cake proteins, and then analyzed them by Western blotting. Total HSL tended to be higher in Lgals12^{-/-} adipocytes (FIG. 9A), which could be partially responsible for the increased lipolysis. Stimulation with isoproterenol resulted in approximately two-fold higher phospho-HSL levels in the fat cake of Lgals^{-/-} adipocytes compared to Lgals12^{+/+} adipocytes (FIGS. 9A and B). Consistent with enhanced translocation of PKA-phosphorylated HSL, the total HSL levels in lipid droplets of Lgals^{-/-} adipocytes were also higher than those in lipid droplets of Lgals12^{+/+} adipocytes after isoproterenol stimulation (FIGS. 9A and B).

Enhanced PKA phosphorylation of HSL was also confirmed by immunofluorescence staining of mouse primary embryonic fibroblast (MEF)-derived adipocytes from Lgals12^{+/+} and Lgals^{-/-} mice (FIG. 9C). We also observed more ATGL associated with lipid droplets in Lgals12−/− adipocytes compared to Lgals12^{+/+} counterparts, with or without isoproterenol stimulation (FIG. 9A, D). There were no significant differences in total ATGL or perilipin levels between adipocytes of the two genotypes (FIG. 9A). These results suggest that increased PKA phosphorylation/activation of adipocyte lipases and their recruitment to lipid droplets account for enhanced lipolysis in Lgals12^{-/-} adipocytes.

Defective PDE Activity Contributes to Enhanced cAMP Levels and Lipolysis in Lgals12−/− Adipocytes.

PKA activity is dynamically regulated by the second messenger cAMP. We compared the intracellular cAMP levels of Lgals12^{+/+} and Lgals12^{-/-} adipocytes before and after stimulation with various concentrations of isoproterenol and found that the cAMP levels in Lgals12^{-/-} adipocytes were significantly higher than in Lgals12^{+/+} adipocytes (FIG. 9E). This suggests that galectin-12 acts upstream of PKA to regulate lipolysis by restricting intracellular cAMP levels.

Intracellular cAMP levels are regulated by stimulatory and inhibitory signaling that regulate adenylyl cyclase activity, as well as enzymes (PDEs) that catalyze its degradation. Treatment with isobutylmethylxanthine (IBMX), which is both a broad-spectrum PDE inhibitor and an adenosine antagonist, greatly enhanced lipolysis in both Lgals12^{+/+} and Lgals12^{-/-} adipocytes and eliminated the observed differences between the two genotypes (FIG. 9F). This result suggests that upstream stimulatory signaling leading to adenylyl cyclase activation does not differ between the two genotypes, and defective tonic anti-lipolytic mechanisms (adenosine signaling or PDE activity) are responsible for enhanced lipolysis in Lgals12^{-/-} adipocytes.

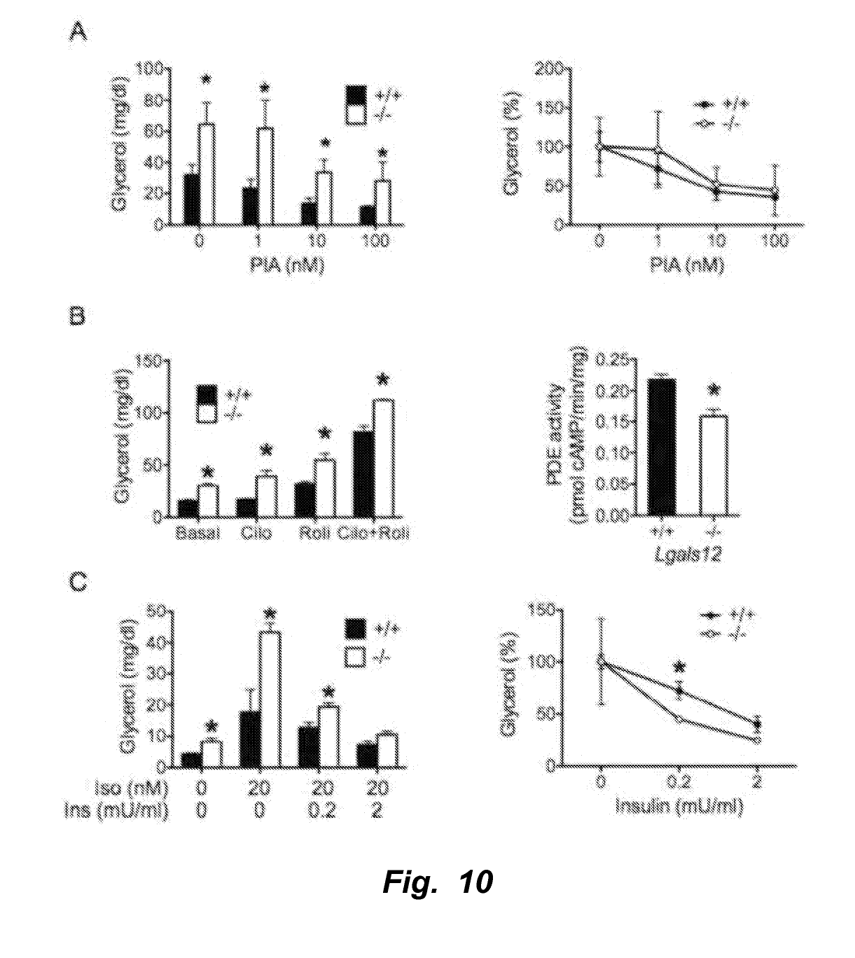

Incubation with adenosine deaminase (ADA), which converts extracellular adenosine to inosine, failed to eliminate differential lipolysis between Lgals12^{+/+} and Lgals^{-/-} cells (FIG. 9F). In the meantime, (−)-N6-(2-Phenylisopropyl)adenosine (PIA), an ADA-resistant A₁ adenosine receptor agonist, suppressed lipolysis in both genotypes with similar efficiencies (FIG. 10A). The results suggest that the adenosine signaling pathway does not account for differential lipolysis in Lgals12^{+/+} and Lgals^{-/-} cells. Instead, defective PDE activity is responsible for enhanced lipolysis in Lgals12^{-/-} adipocytes. This was further confirmed by direct incubation of Lgals12^{+/+} and Lgals12−/− adipocytes with cAMP or its PDE-resistant analog dibutyryl cAMP (dbcAMP), in the presence of the cell-permeable adenylyl cyclase inhibitor, SQ 22536. Incubation with cAMP stimulated greater lipolysis in Lgals12^{-/-} adipocytes than in Lgals12^{+/+} adipocytes, whereas incubation with the PDE-resistant dbcAMP induced comparable lipolysis in adipocytes of the two genotypes (FIG. 9G). Results from experiments with inhibitors of PDE3 and PDE4 suggest that the PDE modulated by galectin-12 in lipolysis is distinct from these two families of phosphodiesterases (FIG. 10B). Consistent with our hypothesis, lipolysis in Lgals12−/− adipocytes remained sensitive to insulin (FIG. 10C), which suppresses lipolysis by activating PDE3B (28,29).

Galectin-12 Deficiency Prevents the Development of Insulin Resistance and Glucose Intolerance Associated with Weight Gain.

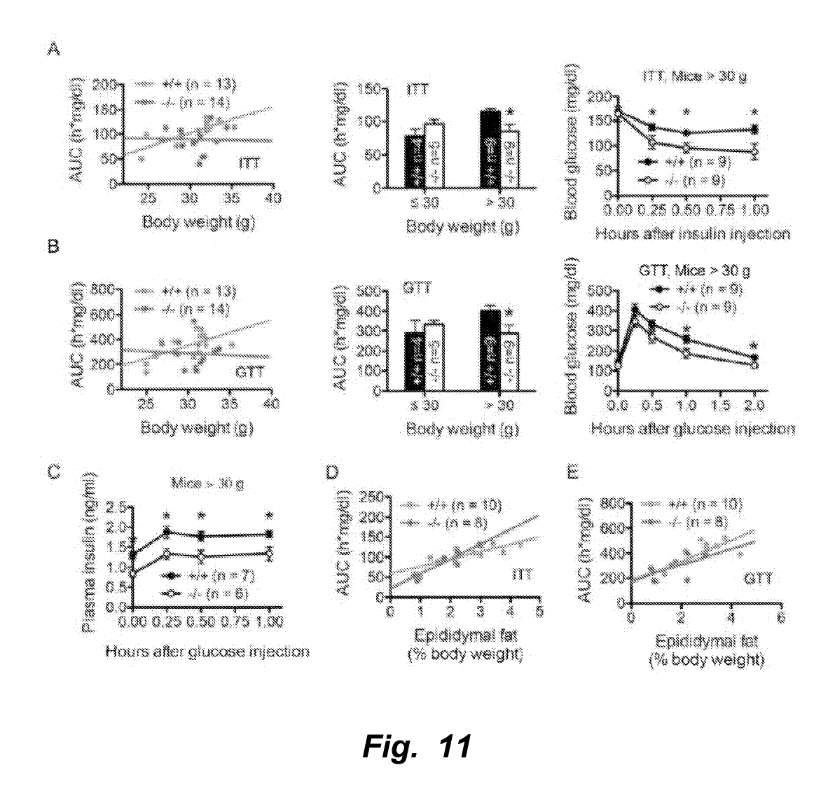

Elevated weight gain and obesity are associated with alterations in adipose tissue functions that predispose an individual to insulin resistance and glucose intolerance preceding the development of type 2 diabetes (13,30). We compared insulin resistance (FIG. 11A) and glucose intolerance (FIG. 11B) of Lgals12^{+/+} and Lgals12^{-/-} mice, as measured by integrating blood glucose levels as a function of time after i.p. injection of insulin or glucose indicated by area under the curve (AUC). In Lgals12^{+/+} mice, insulin resistance and glucose intolerance strongly and positively correlated with body weight. In contrast, no such correlations were observed in Lgals12^{-/-} mice (FIG. 11A, B). Since Lgals12^{+/+} mice >30 g developed insulin resistance and glucose intolerance (FIG. 11A, B, middle panels), we used the 30 g cutoff to test whether galectin-12 ablation improves these parameters, a common practice employed in similar studies (31,32). Results presented in FIGS. 11A and B, middle and right panels, show that galectin-12 deficiency improved insulin sensitivity and glucose tolerance in mice heavier than 30 g. In comparison, perilipin deficiency augmented insulin resistance and glucose intolerance in mice exceeding 30 g, possibly as a result of elevated blood levels of fatty acids that impair insulin sensitivity (32).

Compared to Lgals12^{+/+} animals, improved glucose tolerance in mice >30 g was achieved with lower insulin levels in Lgals12^{-/-} mice (FIG. 9C), consistent with increased insulin sensitivity in these animals. Improved insulin action and glucose homeostasis in Lgals12^{-/-} mice two genotypes (FIG. 11G). Results from experiments with inhibitors of PDE3 and PDE4 suggest that the PDE modulated by galectin-12 in lipolysis is distinct from these two families of phosphodiesterases (FIG. 10B). Consistent with our hypothesis, lipolysis in Lgals12^{-/-} adipocytes remained sensitive to insulin (FIG. 10C), which suppresses lipolysis by activating PDE3B (28,29).

Galectin-12 deficiency prevents the development of insulin resistance and glucose intolerance associated with weight gain. Elevated weight gain and obesity are associated with alterations in adipose tissue functions that predispose an individual to insulin resistance and glucose intolerance preceding the development of type 2 diabetes (13,30). We compared insulin resistance (FIG. 11A) and glucose intolerance (FIG. 11B) of Lgals12^{+/+} and Lgals12^{-/-} mice, as measured by integrating blood glucose levels as a function of time after i.p. injection of insulin or glucose indicated by area under the curve (AUC). In Lgals12^{+/+} mice, insulin resistance and glucose intolerance strongly and positively correlated with body weight. In contrast, no such correlations were observed in Lgals12^{-/-} mice (FIG. 11A, B). Since Lgals12^{+/+} mice >30 g developed insulin resistance and glucose intolerance (FIG. 11A, B, middle panels), we used the 30 g cutoff to test whether galectin-12 ablation improves these parameters, a common practice employed in similar studies (31,32). Results presented in FIGS. 11A and B, middle and right panels, show that galectin-12 deficiency improved insulin sensitivity and glucose tolerance in mice heavier than 30 g. In comparison, perilipin deficiency augmented insulin resistance and glucose intolerance in mice exceeding 30 g, possibly as a result of elevated blood levels of fatty acids that impair insulin sensitivity (32).

Compared to Lgals12^{+/+} animals, improved glucose tolerance in mice >30 g was achieved with lower insulin levels in Lgals12^{-/-} mice (FIG. 11C), consistent with increased insulin sensitivity in these animals. Improved insulin action and glucose homeostasis in Lgals12^{-/-} mice could be the result of reduced adiposity in these mice, or improved adipose tissue function compared with wildtype mice of similar adiposity. Analyses of insulin resistance and glucose intolerance in relation to adiposity in mice revealed similar functional correlations between both genotypes (FIG. 11D, E), suggesting that galectin-12 deficiency enhances insulin responses primarily as a result of reduced adiposity.

### Discussion

Our work identifies galectin-12 as a negative regulator of lipolysis that is preferentially expressed in adipocytes. It is specifically localized on lipid droplets and regulates lipolytic PKA signaling. Galectin-12 deficiency reduces adiposity and prevents development of insulin resistance associated with increased body weight. Thus, we identify a unique intracellular galectin performing a critical function in lipid metabolism that is specifically localized to an organelle.

Galectin-12 ablation altered neither the expression of major adipose genes (FIG. 3), nor the number of adipocytes (FIG. 2) in adipose tissue. This suggests that adipogenesis is largely normal in Lgals12−/− mice, despite previous observations that galectin-12 expression was required for the adipocyte differentiation of 3T3-L1 cells in vitro (15). The absence of an overt adipogenenic phenotype in vivo may be explained by genetic robustness against null mutations in the germline (33). Thus, absence of the adipogenesis phenotype in Lgals12−/− mice is likely the result of functional compensation by another adipogenic pathway.

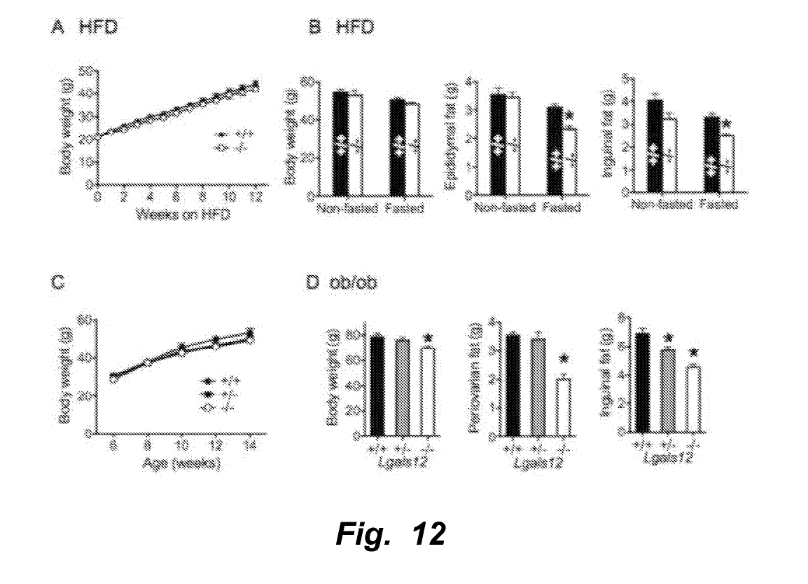

Lgals12^{+/+} and Lgals12^{-/-} mice exhibited similar growth curves when they were fed a high-fat diet for up to 12 weeks (FIG. 12A). Their body weights were indistinguishable after 22 weeks on this diet (FIG. 12B). Similarly, galectin-12 deficiency did not significantly alter adiposity in young leptin-deficient (ob/ob) mice (FIG. 12C). This may be explained by the fact that in both models, obesity develops mainly as a result of excessive food intake. Thus, synthesis of triglycerides in these animals greatly exceeds fat mobilization (lipolysis) and this massive synthesis is likely to marginalize the contribution of lipolysis to the development of increased adiposity. Indeed, in the diet-induced obesity model, a greater reduction of adiposity in ob/ob Lgals12^{-/-} mice was observed after animals were fasted to eliminate the contributions of food/lipid intake and positive energy balance and to simultaneously stimulate lipolysis (FIG. 12B). Similarly, galectin-12 ablation reduced adiposity of ob/ob mice after aging (12 months, FIG. 12D), when hyperphagia lessens.

PKA signaling is characterized by spatiotemporal regulation of signal strength and specificity (34,35). As an example, PKA activation can be regulated locally by compartmentalized PDE activity that degrades cAMP (35,36). Predominant localization of galectin-12 in lipid droplets suggests that it could contribute to such spatial specificity of PKA signaling to lipolytic binding partners on, or around the lipid droplet. Such localized regulation of PKA signaling by galectin-12 is supported by our observation that in wildtype adipocytes, lipid droplets with higher levels of galectin-12 are associated with lower PKA-phosphorylated HSL in response to stimulation by isoproterenol (FIG. 9C).

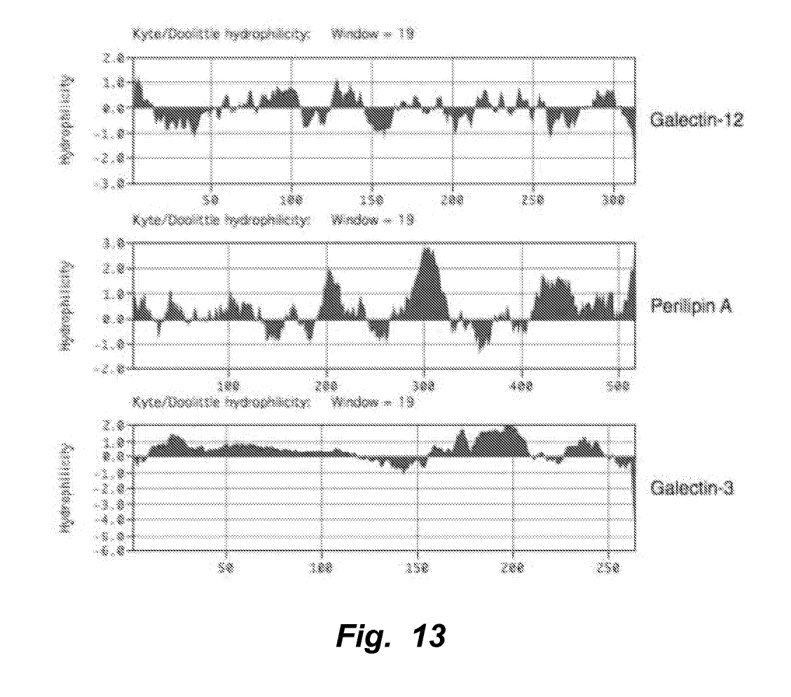

The mechanisms by which the perilipin family of lipid droplet proteins are associated with lipid droplets may be diverse. However, it appears that they all involve hydrophobic interactions (37). Although it is presently unknown how galectin-12 is anchored to lipid droplets, galactosyl glycans are not likely to be involved as the association of galectin-12 to lipid droplets was unaffected by the presence of lactose (FIG. 5). On the other hand, there are several hydrophobic regions in the galectin-12 molecule that may contribute to its localization to lipid droplets (FIG. 13). Lipid domains may also serve as a hydrophobic matrix that helps shape galectin-12 into a functional conformation.

Our results suggest that enhanced lipolysis in Lgals12^{-/-} mice is associated with increased mitochondrial respiration in adipocytes, and elevated whole-body energy expenditure. Since galectin-12 was not found in the mitochondrial fraction, it is not likely that it directly regulates mitochondrial function. Instead, enhanced lipolysis in these cells could lead to elevated levels of intracellular fatty acids that serve both as fuel for the mitochondria and ligands for the PPAR family of transcription factors to activate genes that promote mitochondrial biogenesis (38). In addition, fatty acids have been shown to activate AMP-activated protein kinase (AMPK) that functions to stimulate fatty acid oxidation (39). All these could contribute to higher rates of mitochondrial respiration in Lgals12^{-/-} adipocytes.

Together, the results from these experiments suggest that enhanced lipolysis in Lgals12^{-/-} mice is associated with increased utilization of lipolytic products as fuel for mitochondrial respiration, contributing to higher whole-body energy expenditure and lower adiposity in these mice. Such changes in energy metabolism favor enhanced insulin action in the regulation of glucose homeostasis. From a clinical viewpoint, pharmaceutical targeting of galectin-12 may prove beneficial by both reducing adiposity and improving insulin sensitivity.

In conclusion, we have identified galectin-12, an intracellular form of galectin that is preferentially expressed in adipocytes, as a potential therapeutic target for obesity and associated metabolic conditions, such as insulin resistance and glucose intolerance, that predispose individuals to develop type 2 diabetes.

### Example 2

### Identification of Galectin-12 Inhibitors from Libraries

Illustrative strategies for identifying galectin-12 inhibitors from libraries include without limitation:
1) small organic one-bead-one-compound (OBOC) libraries built around a galactose, lactose or thiodigalactose nucleus to which a polycyclic scaffold is attached, and on which the library of functional groups is connected, and
2) small organic one-bead-one-compound (OBOC) libraries built without a sugar group, but containing a polycyclic scaffold on which the library of functional groups is connected, and
3) modification of existing inhibitors developed for galectin-3 (or other galectins) based on small organic compounds, with or without a sugar core.

With respect to small organic one-bead-one-compound (OBOC) libraries built around a galactose, lactose or thiodigalactose nucleus to which a polycyclic scaffold is attached, and on which the library of functional groups is connected, such compounds contain a sugar group. The general structure of the beads can be described as containing two or three heterocyclic or aromatic groups (R1 and R2 in FIG. 14) conjugated via a scaffold (I), and a galactose residue (II) conjugated via a hydroxy amino acid (III, serine in FIG. 14). Examples of other heterocyclic small molecule libraries (without showing the (φ-[galactose] moiety) are shown in FIG. 15. Generation of small organic one-bead-one-compound (OBOC) libraries is described, e.g., in Aina, et al., Mol Pharm. (2007) 4(5):631-51.

Library Synthesis.

Encoded libraries can be synthesized on TentaGel S NH₂ resin (90 μM, 0.26 mmol/g) with testing compounds displayed on the exterior of the bead and coding molecules reside in the interior. The synthetic routes of the 12 encoded small molecule libraries are outlined in FIG. 15, from which 8 libraries are screened at the rate of two each year. The diversities of each of these libraries can range from 100,000 to 300,000 compounds. A total of over 2 million compounds are synthesized and screened over the 4 year funding period. Libraries 1 to 8 are assembled on a preformed scaffold. In the libraries 9-12, the first amino acid building block (R1) will be encoded separately by carboxylic acids. Synthesis of encoded libraries can be simplified since two layer beads and the linkers can all be prepared and assembled in bulk in advance, and put on the shelf prior to the synthesis of individual libraries. This can save a great deal of time and effort as subsequent steps in the library synthesis can proceed from that point without starting from the beginning each time.

With respect to small organic one-bead-one-compound (OBOC) libraries built without a sugar group, but containing a polycyclic scaffold on which the library of functional groups is connected, the basic structure of the library is depicted in FIG. 16. A structure of a small molecule library without sugar groups is shown: Aa1 is replaced with a linker, but does not contain amino acids, and R2, R3 contain functional groups as indicated. Construction of the library would be similar to that above.

With respect to libraries based on the modification of existing inhibitors developed for galectin-3 (or other galectins) based on small organic compounds, with or without a sugar core, the cyclic functional group-X can be modified to change specificity and selectivity for galectin-12. Exemplary core structures are depicted in FIG. 17. Structure 2 shown contains an N-Ac-lactosamine core; structure 4 contains a thiodigalactose core. See, e.g., Cumpstey, et al., Angew Chem Int Ed Engl. (2005) 44(32):5110-2 and Sorme, et al., Chembiochem. (2002) 3(2-3):183-9.

### REFERENCES

1. Cummings R D, Liu F (2009) in Essentials of Glycobiology, eds Varki A, Cummings R D, Esko J D, Freeze H H, Stanley P, Bertozzi C R, Hart G W, Etzler M E (Cold Spring Harbor Laboratory Press, Cold Spring Harbor, N.Y.), pp 475-488.
2. Wang J L, Gray R M, Haudek K C, Patterson R J (2004) Nucleocytoplasmic lectins. Biochim Biophys Acta 1673:75-93.
3. Liu F T, Rabinovich G A (2005) Galectins as modulators of tumour progression. Nat Rev Cancer 5:29-41.
4. Yang R Y, Rabinovich G A, Liu F T (2008) Galectins: structure, function and therapeutic potential. Expert Rev Mol Med 10:e17.
5. Nakahara S, Oka N, Raz A (2005) On the role of galectin-3 in cancer apoptosis. Apoptosis 10:267-275.
6. Thijssen V L, Poirier F, Baum L G, Griffioen A W (2007) Galectins in the tumor endothelium: opportunities for combined cancer therapy. Blood 110:2819-2827.
7. Rabinovich G A, Toscano M A (2009) Turning ‘sweet’ on immunity: galectin-glycan interactions in immune tolerance and inflammation. Nat Rev Immunol 9:338-352.
8. Yang R Y, Hsu D K, Yu L, Ni J, Liu F T (2001) Cell cycle regulation by galectin-12, a new member of the galectin superfamily. J Biol Chem 276:20252-20260.
9. Hotta K et al. (2001) Galectin-12, an Adipose-expressed Galectin-like Molecule Possessing Apoptosis-inducing Activity. J Biol Chem 276:34089-34097.
10. Kouadjo K E, Nishida Y, Cadrin-Girard J F, Yoshioka M, St-Amand J (2007) Housekeeping and tissue-specific genes in mouse tissues. BMC Genomics 8:127.
11. Martin S, Parton R G (2006) Lipid droplets: a unified view of a dynamic organelle. Nat Rev Mol Cell Biol 7:373-378.
12. Duncan R E, Ahmadian M, Jaworski K, Sarkadi-Nagy E, Sul H S (2007) Regulation of lipolysis in adipocytes. Annu Rev Nutr 27:79-101.
13. Guilherme A, Virbasius J V, Puri V, Czech M P (2008) Adipocyte dysfunctions linking obesity to insulin resistance and type 2 diabetes. Nat Rev Mol Cell Biol 9:367-377.
14. Fasshauer M, Klein J, Lossner U, Paschke R (2002) Negative regulation of adiposeexpressed galectin-12 by isoproterenol, tumor necrosis factor alpha, insulin and dexamethasone. Eur J Endocrinol 147:553-559.
15. Yang R Y, Hsu D K, Yu L, Chen H Y, Liu F T (2004) Galectin-12 is required for adipogenic signaling and adipocyte differentiation. J Biol Chem 279:29761-29766.
16. Havel P J (2004) Update on adipocyte hormones: regulation of energy balance and carbohydrate/lipid metabolism. Diabetes 53 Suppl 1:S143-S151.
17. Piper R C, Hess L J, James D E (1991) Differential sorting of two glucose transporters expressed in insulin-sensitive cells. Am J Physiol 260:C570-0580.
18. Brasaemle D L, Subramanian V, Garcia A, Marcinkiewicz A, Rothenberg A (2008) Perilipin A and the control of triacylglycerol metabolism. Mol Cell Biochem 326:15-21.
19. Moore H P, Silver R B, Mottillo E P, Bernlohr D A, Granneman J G (2005) Perilipin targets a novel pool of lipid droplets for lipolytic attack by hormone-sensitive lipase. J Biol Chem 280:43109-43120.
20. Toh S Y et al. (2008) Up-regulation of mitochondrial activity and acquirement of brown adipose tissue-like property in the white adipose tissue of fsp27 deficient mice. PLoS One 3:e2890.
21. Nishino N et al. (2008) FSP27 contributes to efficient energy storage in murine white adipocytes by promoting the formation of unilocular lipid droplets. J Clin Invest 118:2808-2821.
22. Singh R et al. (2009) Autophagy regulates adipose mass and differentiation in mice. J Clin Invest 119:3329-3339.
23. Krintel C et al. (2008) Ser649 and Ser650 are the major determinants of protein kinase A mediated activation of human hormone-sensitive lipase against lipid binding partners. PLoS ONE 3:e3756.
24. Granneman J G et al. (2007) Analysis of lipolytic protein trafficking and interactions in adipocytes. J Biol Chem 282:5726-5735.
25. Haemmerle G et al. (2006) Defective lipolysis and altered energy metabolism in mice lacking adipose triglyceride lipase. Science 312:734-737.
26. Zimmermann R et al. (2004) Fat mobilization in adipose tissue is promoted by adipose triglyceride lipase. Science 306:1383-1386.
27. Haemmerle G et al. (2002) Hormone-sensitive lipase deficiency in mice causes diglyceride accumulation in adipose tissue, muscle, and testis. J Biol Chem 277:4806-4815.
28. Kitamura T et al. (1999) Insulin-induced phosphorylation and activation of cyclic nucleotide phosphodiesterase 3B by the serine-threonine kinase Akt. Mol Cell Biol 19:6286-6296.
29. Choi Y H et al. (2006) Alterations in regulation of energy homeostasis in cyclic nucleotide phosphodiesterase 3B-null mice. J Clin Invest 116:3240-3251.
30. Lazar M A (2005) How obesity causes diabetes: not a tall tale. Science 307:373-375.
31. Banerjee R R et al. (2004) Regulation of fasted blood glucose by resistin. Science 303:1195-1198.
32. Tansey J T et al. (2001) Perilipin ablation results in a lean mouse with aberrant adipocyte lipolysis, enhanced leptin production, and resistance to diet-induced obesity. Proc Natl Acad Sci USA 98:6494-6499.
33. Wagner A (2005) Distributed robustness versus redundancy as causes of mutational robustness. Bioessays 27:176-188.
34. Jarnaess E, Tasken K (2007) Spatiotemporal control of cAMP signalling processes by anchored signalling complexes. Biochem Soc Trans 35:931-937.
35. Zaccolo M, Pozzan T (2002) Discrete microdomains with high concentration of cAMP in stimulated rat neonatal cardiac myocytes. Science 295:1711-1715.
36. Lehnart S E et al. (2005) Phosphodiesterase 4D deficiency in the ryanodine-receptor complex promotes heart failure and arrhythmias. Cell 123:25-35.
37. Brasaemle D L (2007) Thematic review series: adipocyte biology. The perilipin family of structural lipid droplet proteins: stabilization of lipid droplets and control of lipolysis. J Lipid Res 48:2547-2559.
38. Puri V, Czech M P (2008) Lipid droplets: FSP27 knockout enhances their sizzle. J Clin Invest 118:2693-2696.
39. Long Y C, Zierath J R (2006) AMP-activated protein kinase signaling in metabolic regulation. J Clin Invest 116:1776-1783.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of promoting lipolysis and/or reducing adiposity in a subject, comprising administering to the subject an effective amount of an inhibitor of galectin-12 activity, thereby promoting lipolysis and/or reducing adiposity in the subject.

2. A method of promoting and/or increasing insulin sensitivity and/or glucose tolerance in a subject, comprising administering to the subject an effective amount of an inhibitor of galectin-12 activity, thereby promoting and/or increasing insulin sensitivity and/or glucose tolerance in the subject.

3. The method of claim 1, wherein the subject is obese.

4. The method of claim 1, wherein the subject has type 2 diabetes.

5. The method of claim 1, wherein the subject has metabolic disease.

6. The method of claim 1, wherein the subject has cardiovascular disease.

7. (canceled)

8. A method of preventing, inhibiting, mitigating, or delaying one or more symptoms of a mitochondrial disease in a subject, comprising administering to the subject an effective amount of an inhibitor of galectin-12 activity, thereby preventing, inhibiting, mitigating, or delaying one or more symptoms of the mitochondrial disease by promoting and/or increasing mitochondrial respiration in the subject.

9. (canceled)

10. The method of claim 8, wherein the subject has a mitochondrial disease resulting from dysfunctional mitochondria in cells wherein galectin-12 is constitutively expressed or abnormally overexpressed.

11. The method of claim 8, wherein the subject has a mitochondrial disease selected from the group consisting of Luft disease, Leigh syndrome (Complex I, cytochrome oxidase (COX) deficiency, pyruvate dehydrogenase (PDH) deficiency), Alpers Disease, Medium-Chain Acyl-CoA Dehydrongenase Deficiency (MCAD), Short-Chain Acyl-CoA Dehydrogenase Deficiency (SCAD), Short-chain-3-hydroxyacyl-CoA dehydrogenase (SCHAD) deficiency, Very Long-Chain Acyl-CoA Dehydrongenase Deficiency (VLCAD), Long-chain 3-hydroxyacyl-CoA dehydrogenase (LCHAD) deficiency, glutaric aciduria II, lethal infantile cardiomyopathy, Friedreich ataxia, maturity onset diabetes of young, malignant hyperthermia, disorders of ketone utilization, mtDNA depletion syndrome, reversible cox deficiency of infancy, various defects of the Krebs cycle, pyruvate dehydrogenase deficiency, pyruvate carboxylase deficiency, fumarase deficiency, carnitine palmitoyl transferase deficiency.

12. The method of claim 8, wherein the subject has a cancer associated with or caused by the constitutive expression or overexpression of galectin-12.

13. (canceled)

14. The method of claim 1, wherein the inhibitor of galectin-12 activity inhibits the binding of galectin-12 to beta-galactose-containing ligands or its proteinaceous binding partners.

15. (canceled)

16. The method of claim 14, wherein the inhibitor of galectin-12 activity inhibits the binding of galectin-12 to one or more proteinaceous binding partners selected from the group consisting of mitochondrial chaperone HSP60, heat-shock cognate 70 (Hsc70), and vacuolar protein sorting 13 (VPS13).

17. The method of claim 14, wherein the inhibitor of galectin-12 activity is a glycan mimetic.

18. The method of claim 14, wherein the inhibitor of galectin-12 activity is a peptide.

19. The method of claim 14, wherein the inhibitor of galectin-12 activity is an antigen binding molecule.

20. The method of claim 14, wherein the inhibitor of galectin-12 activity is an inhibitory nucleic acid.

21. (canceled)

22. The method of claim 14, wherein the inhibitor of galectin-12 activity comprises a substituted core comprised of a galactose, lactose, an oligo-lactose, a poly-lactose, thiodigalactose, N-Acetyl-lactosamine or analogs and/or derivatives thereof, attached to a scaffold comprising one or more linear, cyclic, aromatic, polycyclic linkers as depicted in FIG. 14, FIG. 15A, FIG. 15B, FIG. 16, or FIG. 17.

23-25. (canceled)

26. The method of claim 1, wherein the inhibitor of galectin-12 activity is administered orally, intravenously, topically, transdermally, or delivered to its site of action directly or remotely.

27. The method of claim 1, wherein the inhibitor of galectin-12 activity inhibits the expression of galectin-12.

28-30. (canceled)

31. The method of claim 1, wherein the subject is a human.

32-33. (canceled)
